(19) United States
(12) Patent Application Publication
(10) Pub. No.: US 2004/0157863 A1
(43) Pub. Date: Aug. 12, 2004

(54) Triazolopyrimidines

(76) Inventors: Olaf Gebauer, Koln (DE); Jorg Nico Greul, Leichligen (DE); Ulrich Heinemann, Leichlingen (DE); Hans-Ludwig Elbe, Wuppertal (DE); Bernd-Wieland Kruger, Berglach Gladbach (DE); Ralf Dunkel, Monheim (DE); Arnd Voerste, Koln (DE); Ronald Ebbert, Monheim (DE); Ulrike Wachendorff-Neumann, Neuwied (DE); Karl-Heinz Kuck, Langenfeld (DE); Yoshinori Kitagawa, Tochigi (JP)

Correspondence Address: Bayer CropScience, Patent Department, 100 Bayer Road, Pittsburgh, PA, 15205-9741, US

(21) Appl. No.: 10/474,948

(22) PCT Filed: Apr. 23, 2002
(86) PCT No.: PCT/EP02/04441

(30) Foreign Application Priority Data
Apr. 27, 2001 (DE) 101 21 102.3

(51) Int. Cl. A61K 31/519; C07D 487/02

(52) U.S. Cl. 514/259.31; 544/263

(57) ABSTRACT

Novel triazolopyrimidines of the formula

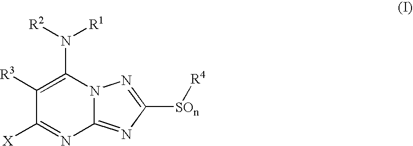

in which

R¹, R², R³, R⁴, X and n have the meanings given in the description,

a plurality of processes for preparing these novel substances and their use for controlling unwanted microorganisms.

Novel intermediates of the formulae given in the description and processes for the preparation of these intermediates.

The present invention relates to novel triazolopyrimidines, to a plurality of processes for their preparation and to their use for controlling unwanted microorganisms. The invention furthermore relates to novel intermediates and to processes for their preparation.

It is already known that certain triazolopyrimidines have fungicidal properties (cf. EP-A 0 550 113, WO 94-20 501, EP-A 0 613 900, US-A 5 612 345, EP-A 0 834 513, WO 98-46 607 and WO 98-46 608). The activity of these substances is good; however, at low application rates, it is sometimes unsatisfactory.

This invention, accordingly, provides novel triazolopyrimidines of the formula

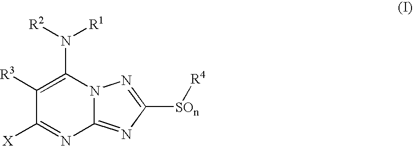

in which

R¹ represents amino, optionally substituted alkyl, optionally substituted alkenyl, optionally substituted alkinyl, optionally substituted cycloalkyl, optionally substituted alkoxy, optionally substituted alkenyloxy, optionally substituted alkinyloxy, optionally substituted cycloalkyloxy, optionally substituted alkylamino, optionally substituted dialkylamino, optionally substituted alkenylamino, optionally substituted alkinylamino, optionally substituted cycloalkylamino, optionally substituted N-cycloalkyl-N-alkylamino, optionally substituted alkylideneamino, optionally substituted heterocyclyl or represents a radical of the formula —S—R⁵, in which

R⁵ represents optionally substituted alkyl, optionally substituted alkenyl, optionally substituted alkinyl or optionally substituted cycloalkyl,

R² represents hydrogen, optionally substituted alkyl, optionally substituted alkenyl, optionally substituted alkinyl, or optionally substituted cycloalkyl,

or

R¹ and R² together with the nitrogen atom to which they are attached represent an optionally substituted heterocyclic ring,

R³ represents optionally substituted aryl,

R⁴ represents optionally substituted alkyl, optionally substituted alkenyl or optionally substituted alkinyl,

X represents halogen and

n represents 0, 1 or 2,

and acid addition salts of those compounds of the formula (1)

in which

R¹ represents amino.

Depending on substitution pattern the compounds of the invention may optionally be present as mixtures of different possible isomeric forms, particularly of stereoisomers, such as E and Z isomers, threo and erythro isomers, and optical isomers, for example, and optionally of tautomers. Where R³ is substituted differently on both atoms adjaent to the bonding site, the compounds in question may be present in one particular form of stereoisomerism, namely as atropisomers.

Furthermore, it has been found that triazolopyrimidines of the formula (I) can be prepared by

a) reacting dihalogeno-triazolopyrimidines of the formula

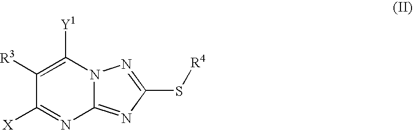

in which

R³, R⁴ and X have the meanings given above and

Y¹ represents halogen

with amines of the formula

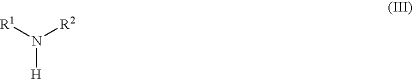

in which

R¹ and R² have the meanings given above,

if appropriate in the presence of a diluent and if appropriate in the presence of an acid acceptor,

or

b) reacting triazolopyrimidines of the formula

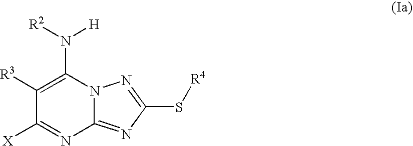

in which

R², R³, R⁴ and X have the meanings given above

with sulphenyl halides of the formula

Y²—S—R⁵ (IV)

in which

R⁵ has the meanings given above and

Y² represents halogen,

if appropriate in the presence of a diluent and if appropriate in the presence of an acid acceptor,

or

c) reacting triazolopyrimidines of the formula

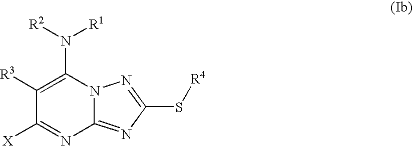

in which

R¹, R², R³, R⁴ and X have the meanings given above,

with oxygen-releasing oxidizing agents, if appropriate in the presence of a diluent and if appropriate in the presence of a catalyst,

and if appropriate adding an acid to the resulting compounds of the formula (I)

in which

R¹ represents amino.

Finally, it has been found that the novel triazolopyrimidines of the formula (1) and their acid addition salts are highly suitable for controlling unwanted microorganisms. In particular, they have strong fungicidal activity and can be used both in crop protection and in the protection of materials.

Surprisingly, the triazolopyrimidines of the formula (1) according to the invention have considerably better microbicidal activity than the constitutionally most similar prior-art substances of the same direction of action.

The formula (I) provides a general definition of the triazolopyrimidines according to the invention.

R¹ preferably represents amino;

represents alkyl having 1 to 6 carbon atoms which is optionally substituted by halogen, cycloalkyl, cyano, phenyl, heterocyclyl, alkoxy having 1 to 4 carbon atoms, alkylthio having 1 to 4 carbon atoms, oxo, hydroxoimino and/or alkoximino having 1 to 4 carbon atoms,

represents alkyl having 1 to 6 carbon atoms which is optionally substituted by hydroxy, alkenyloxy having 2 to 4 carbon atoms, alkoxycarbonyl having 1 to 4 carbon atoms,

represents alkenyl having 2 to 6 carbon atoms which is optionally substituted by halogen, cycloalkyl, cyano, phenyl and/or heterocyclyl,

represents alkinyl having 2 to 6 carbon atoms which is optionally substituted by halogen, cycloalkyl, cyano, phenyl and/or heterocyclyl,

represents cycloalkyl having 3 to 7 carbon atoms which is optionally substituted by halogen, cycloalkyl, cyano, phenyl and/or heterocyclyl,

represents alkoxy having 1 to 7 carbon atoms which is optionally substituted by halogen, cycloalkyl, cyano, phenyl and/or heterocyclyl,

represents alkenyloxy having 2 to 6 carbon atoms which is optionally substituted by halogen, cycloalkyl, cyano, phenyl and/or heterocyclyl,

represents alkinyloxy having 2 to 6 carbon atoms which is optionally substituted by halogen, cycloalkyl, cyano, phenyl and/or heterocyclyl,

represents cycloalkyloxy having 3 to 7 carbon atoms which is optionally substituted by halogen, cycloalkyl, cyano, phenyl and/or heterocyclyl,

represents alkylamino having 1 to 7 carbon atoms which is optionally substituted by halogen, cycloalkyl, cyano, phenyl and/or heterocyclyl,

represents dialkylamino having 1 to 7 carbon atoms in each of the alkyl radicals which is optionally substituted by halogen, cycloalkyl, cyano, phenyl and/or heterocyclyl,

represents alkenylamino having 2 to 6 carbon atoms which is optionally substituted by halogen, cycloalkyl, cyano, phenyl and/or heterocyclyl,

represents alkylamino having 2 to 6 carbon atoms which is optionally substituted by halogen, cycloalkyl, cyano, phenyl and/or heterocyclyl,

represents cycloalkylamino having 3 to 7 carbon atoms which is optionally substituted by halogen, cycloalkyl, cyano, phenyl and/or heterocyclyl,

represents N-cycloalkyl-N-alkyl-amino having 3 to 7 carbon atoms in the cycloalkyl moiety and 1 to 7 carbon atoms in the alkyl moiety which is optionally substituted by halogen, cycloalkyl, cyano, phenyl and/or heterocyclyl,

represents alkylideneamino having 2 to 6 carbon atoms which is optionally substituted by halogen, cycloalkyl, cyano, phenyl and/or heterocyclyl,

represents heterocyclyl having 5 or 6 ring members which is optionally substituted by halogen, alkyl, cycloalkyl, cyano, phenyl and/or heterocyclyl or represents —S—R⁵, in which

R⁵ represents alkyl having 1 to 6 carbon atoms which is optionally substituted by halogen, cycloalkyl, cyano, phenyl and/or heterocyclyl,

represents alkenyl having 2 to 6 carbon atoms which is optionally substituted by halogen, cycloalkyl, cyano, phenyl and/or heterocyclyl,

represents alkinyl having 2 to 6 carbon atoms which is optionally substituted by halogen, cycloalkyl, cyano, phenyl and/or heterocyclyl or

represents cycloalkyl having 3 to 7 carbon atoms which is optionally substituted by halogen, cycloalkyl, cyano, phenyl and/or heterocyclyl,

where the heterocyclyl radicals mentioned above may be mono- to trisubstituted by identical or different substituents from the group consisting of halogen, alkyl having 1 to 4 carbon atoms, halogenoalkyl having 1 or 2 carbon atoms and 1 to 5 halogen atoms, alkoxy having 1 to 4 carbon atoms, alkylthio having 1 to 4 carbon atoms, halogenoalkoxy having 1 or 2 carbon atoms and 1 to 5 halogen atoms, halogenoalkylthio having 1 or 2 carbon atoms and 1 to 5 halogen atoms and/or phenyl,

and where the phenyl radicals mentioned above may be mono- to trisubstituted by identical or different substituents from the group consisting of

halogen, cyano, nitro, amino, hydroxyl, formyl, carboxyl, carbamoyl, thiocarbamoyl;

in each case straight-chain or branched alkyl, alkoxy, alkylthio, alkylsulphinyl or alkylsulphonyl having in each case 1 to 6 carbon atoms;

in each case straight-chain or branched alkenyl or alkenyloxy having in each case 2 to 6 carbon atoms;

in each case straight-chain or branched halogenoalkyl, halogenoalkoxy, halogenoalkylthio, halogenoalkylsulphinyl or halogenoalkylsulphonyl having in each case 1 to 6 carbon atoms and 1 to 13 identical or different halogen atoms; in each case straight-chain or branched halogenoalkenyl or halogenoalkenyloxy having in each case 2 to 6 carbon atoms and 1 to 13 identical or different halogen atoms;

in each case straight-chain or branched alkylamino, dialkylamino, alkylcarbonyl, alkylcarbonyloxy, alkoxycarbonyl, alkylsulphonyloxy, hydroximinoalkyl or alkoximinoalkyl having in each case 1 to 6 carbon atoms in the individual alkyl moieties;

cycloalkyl having 3 to 6 carbon atoms,

or may be monosubstituted by alkylene having 3 or 4 carbon atoms or dioxyalkylene having 1 or 2 carbon atoms, where these radicals are doubly attached in the ortho position and may be mono- to tetrasubstituted by identical or different substituents from the group consisting of halogen, alkyl having 1 to 4 carbon atoms and/or halogenoalkyl having 1 to 4 carbon atoms and 1 to 9 identical or different halogen atoms,

R² preferably represents hydrogen,

represents alkyl having 1 to 4 carbon atoms which is optionally substituted by halogen, alkoxy having 1 to 4 carbon atoms, alkylthio having 1 to 4 carbon atoms, oxo, hydroximino, alkoximino having 1 to 4 carbon atoms and/or cycloalkyl having 3 to 6 carbon atoms,

represents alkenyl having 2 to 4 carbon atoms which is optionally substituted by halogen and/or cycloalkyl having 3 to 6 carbon atoms or

represents alkinyl having 2 to 4 carbon atoms which is optionally substituted by halogen and/or cycloalkyl having 3 to 6 carbon atoms or

represents cycloalkyl having 3 to 6 carbon atoms which is optionally substituted by halogen and/or alkyl having 1 to 4 carbon atoms,

R¹ and R² preferably together with the nitrogen atom to which they are attached represent a 3- to 6-membered heterocyclic ring which may be mono- to trisubstituted by identical or different substituents from the group consisting of

alkyl having 1 to 4 carbon atoms,

halogenoalkyl having 1 to 4 carbon atoms and 1 to 9 identical or different halogen atoms,

fluorine, chlorine, bromine, iodine, hydroxyl, cyano, oxo;

alkylcarbonyl having 1 to 4 carbon atoms,

alkoxycarbonyl having 1 to 4 carbon atoms,

aminoalkyl having 1 to 4 carbon atoms,

alkylcarbonylamino having 1 to 4 carbon atoms or

to which a phenyl ring is fused.

R³ preferably represents phenyl which may be mono- to pentasubstituted by identical or different substituents from the group consisting of

halogen, cyano, nitro, amino, hydroxyl, formyl, carboxyl, carbamoyl, thiocarbamoyl;

in each case straight-chain or branched alkyl, alkoxy, alkylthio, alkylsulphinyl or alkylsulphonyl having in each case 1 to 6 carbon atoms;

in each case straight-chain or branched alkenyl or alkenyloxy having in each case 2 to 6 carbon atoms;

in each case straight-chain or branched halogenoalkyl, halogenoalkoxy, halogenoalkylthio, halogenoalkylsulphinyl or halogenoalkylsulphonyl having in each case 1 to 6 carbon atoms and 1 to 13 identical or different halogen atoms;

in each case straight-chain or branched halogenoalkenyl or halogenoalkenyloxy having in each case 2 to 6 carbon atoms and 1 to 13 identical or different halogen atoms;

in each case straight-chain or branched alkylamino, dialkylamino, alkylcarbonyl, alkylcarbonyloxy, alkoxycarbonyl, alkylsulphonyloxy, hydroximinoalkyl or alkoximinoalkyl having in each case 1 to 6 carbon atoms in the individual alkyl, moieties;

cycloalkyl having 3 to 6 carbon atoms,

or may be monosubstituted by alkylene having 3 or 4 carbon atoms or dioxyalkylene having 1 or 2 carbon atoms, where these radicals are doubly attached in the ortho position and may be mono- to tetrasubstituted by identical or different substituents from the group consisting of halogen, alkyl having 1 to 4 carbon atoms and/or halogenoalkyl having 1 to 4 carbon atoms and 1 to 9 identical or different halogen atoms,

R⁴ preferably represents alkyl having 1 to 6 carbon atoms which is optionally substituted by halogen and/or cyano,

represents alkenyl having 3 to 6 carbon atoms which is optionally substituted by halogen and/or cyano,

represents alkinyl having 3 to 6 carbon atoms which is optionally substituted by halogen and/or cyano or

represents aralkyl having 6 to 10 carbon atoms in the aryl moiety and 1 to 4 carbon atoms in the alkyl moiety and being optionally substituted by halogen,

X preferably represents fluorine, chlorine or bromine.

n preferably represents 0, 1 or 2.

R¹ preferably represents methyl, ethyl, n-propyl, isopropyl, n-, iso-, s- or t-butyl, methoxymethyl, 2-methoxy-ethyl, methylthio-methyl, 2-methylthio-methyl, hydroximino-methyl, methoximino-methyl, acetylmethyl, 2-(hydroximino)-propyl, 2-(methoximino)propyl,

or

represents 2-ethoxycarbonyl-ethyl, 2-vinyloxy-ethyl, 2-hydroxyimino-ethyl, 2-methoxyimino-ethyl, 2-hydroxyethyl, 2-chloroethyl, 2-methyl-1-propyl, 1,2-dimethylpropyl, thienylmethyl, amino;

or

represents allyl, 2-methyl-prop-2-enyl, propargyl, 2,2,2-trifluoroethyl, 1-(trifluoromethyl)-ethyl, 3,3,3-trifluoropropyl, cyclopentyl, cyclohexyl, cyclopropylmethyl, cyclobutylmethyl, cyclopentylmethyl or cyclohexylmethyl, methoxy, ethoxy, n- or isopropoxy, n-, iso-, s- or t-butoxy, allyloxy, propargyloxy, cyclopropyloxy, cyclobutyloxy, cyclopentyloxy or cyclohexyloxy, difluoromethoxy, trifluoromethoxy, difluorochloromethoxy, trifluoroethoxy, methylamino, ethylamino, n- or isopropylamino, n-, iso-, s- or t-butylamino, dimethylamino, diethylamino, trifluoroethylamino, cyclohexylmethylamino, 2-cyanoethylamino, allylamino, 1-cyclopropylethylamino, cyclopropylamino, cyclobutylamino, cyclopentylamino, cyclohexylamino, 1-methylethylideneamino, benzyloxy, piperidinyl, morpholinyl, pyridylmethoxy, thiazolylmethoxy or represents —S—R⁵, in which

R⁵ represents methyl, ethyl, n- or isopropyl, difluoromethyl, difluorochloromethyl, dichlorofluoromethyl or trifluoromethyl,

where the thiazolyl and pyridyl radicals mentioned above may in each case be substituted, in the case of thiazolyl mono- or disubstituted and in the case of pyridyl mono- to trisubstituted, by identical or different substituents from the group consisting of fluorine, chlorine, bromine, methyl, ethyl, n- or isopropyl, n- iso-, s- or t-butyl, methoxy, ethoxy, n- or isopropoxy, n-, iso-, s- or t-butoxy, methylthio, ethylthio, n- or isopropylthio, difluoromethoxy, trifluoromethoxy, difluorochloromethoxy, trifluoroethoxy, difluoromethylthio, difluorochloromethylthio, dichlorofluoromethylthio, trifluoromethylthio and phenyl,

and where the benzyloxy mentioned above may be mono- to trisubstituted in the phenyl moiety by identical or different substituents from the group consisting of

fluorine, chlorine, bromine, cyano, nitro, amino, hydroxyl, formyl, carboxyl, carbamoyl, thiocarbamoyl, methyl, ethyl, n- or i-propyl, n-, iso-, s- or t-butyl, methoxy, ethoxy, n- or i-propoxy, methylthio, ethylthio, n- or i-propylthio, methylsulphinyl, ethylsulphinyl, methylsulphonyl or ethylsulphonyl, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, methylamino, ethylamino, n- or i-propylamino, dimethylamino, diethylamino, acetyl, propionyl, acetyloxy, methoxycarbonyl, ethoxycarbonyl, methylsulphonyloxy, ethylsulphonyloxy, hydroximinomethyl, hydroximinoethyl, methoximinomethyl, ethoximinomethyl, methoximinoethyl, ethoximinoethyl, cyclopropyl, cyclobutyl, cyclopentyl or cyclohexyl,

or may be monosubstituted by propene-1,3-diyl, methylenedioxy or ethylenedioxy, where these radicals are doubly attached in the ortho-position and may be mono- to tetrasubstituted by identical or different substituents from the group consisting of fluorine, chlorine, methyl, ethyl, n-propyl, isopropyl and/or trifluoromethyl,

R² particularly preferably represents hydrogen, methyl, ethyl, n- or isopropyl, n-, iso-, s- or t-butyl, cyclopropyl, methoxy-methyl, 2-methoxy-ethyl, methylthiomethyl, 2-methylthio-methyl, hydroximino-methyl, methoximino-methyl, acetylmethyl, 2-hydroximino-propyl, 2-methoximino-propyl

or

represents allyl, propargyl, 2,2,2-trifluoroethyl, 1-(trifluoromethyl)ethyl, cyclopropylmethyl, cyclobutylmethyl, cyclopentylmethyl or cyclohexylmethyl.

R¹ and R² particularly preferably together with the nitrogen atom to which they are attached

represent optionally methyl-, ethyl- or trifluoroethyl-substituted pyrrolyl, piperidinyl, morpholinyl or piperazinyl,

represents piperidinyl which is mono- to trisubstituted by fluorine, chlorine, bromine, hydroxyl, oxo, methoxy, amino, methylamino, dimethylamino, acetylamino, chloromethyl, dichloromethyl, fluoromethyl, trifluoromethyl, aminomethyl, methoxycarbonyl, methylcarbonyl, morpholinyl, 1,3-dioxolanyl and/or tert-butoxycarbonylamino,

represents piperidinyl fused with phenyl,

represents pyrrolidinyl which is unsubstituted or mono- to trisubstituted by hydroxyl, dimethylamino, acetylamino and/or 1,3-propanediyl,

represents unsubstituted or methyl-substituted piperazine, tetrahydrothiazine or tetrahydropyridine.

R³ particularly preferably represents phenyl which may be mono- to trisubstituted by identical or different substituents from the group consisting of

fluorine, chlorine, bromine, cyano, nitro, formyl, methyl, ethyl, n- or isopropyl, n-, iso-, s- or t-butyl, allyl, propargyl, methoxy, ethoxy, n- or isopropoxy, methylthio, ethylthio, n- or isopropylthio, methylsulphinyl, ethylsulphinyl, methylsulphonyl, ethylsulphonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, trichloroethinyloxy, trifluoroethinyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl, ethoximinoethyl, cyclopropyl, cyclobutyl, cyclopentyl and cyclohexyl,

or may be monosubstituted by propane-1,3-diyl, methylenedioxy or ethylenedioxy, where these radicals are doubly attached in the ortho-position and may be mono- to tetrasubstituted by identical: or different substituents from the group consisting of fluorine, chlorine, methyl, ethyl, n-propyl, isopropyl and trifluoromethyl,

R⁴ particularly preferably represents methyl, ethyl, n- or isopropyl, n-, iso-, s- or t-butyl, allyl, propargyl, benzyl or chlorobenzyl,

X particularly preferably represents fluorine or chlorine.

Very particular preference is given to those compounds of the formula (I) in which

R¹, R², R⁴, X and n have the meanings given above and

R³ represents 2-substituted, 2,4-disubstituted, 2,6-disubstituted or 2,4,6-trisubstituted phenyl.

Very particular preference is given to compounds of the formula (I) in which

R⁴ represents methyl and

X represents chlorine and

R¹, R², R³ and n have the meanings given above.

Very particular preference is given to compounds of the formula (I) in which

R¹ and R², together with the nitrogen atom to which they are attached, represent optionally substituted piperidinyl, the possible substituents being already mentioned above, and

R³, R⁴, X and n have the meanings given above.

Very particular preference is given to compounds of the formula (I) in which

R² represents hydrogen, methyl, ethyl or isopropyl and

R¹, R³, R⁴, X and n have one of the meanings given above.

The radical definitions mentioned above can be combined with one another as desired. Moreover, individual meanings may not apply.

Further preferred compounds according to the invention are adducts of acids and those triazolopyrimidines of the formula (I) in which

R¹ represents amino and

R², R³, R⁴, X and n have those meanings stated as being preferred for these radicals and for the index n.

The acids which can be used for addition include preferably hydrohalic acids, such as hydrochloric acid and hydrobromic acid, for example, especially hydrochloric acid, and also phosphoric acid, nitric acid, monofunctional and difunctional carboxylic acids and hydroxycarboxylic acids, such as acetic acid, maleic acid, succinic acid, fumaric acid, tartaric acid, citric acid, salicylic acid, sorbic acid and lactic acid, for example, and sulphonic acids, such as p-toluenesulphonic acid, 1,5-naphthalenedisulphonic acid, saccharin and thiosaccharin, for example.

Using 5,7-dichloro-2-(methylsulphanyl)-6-(2,4,6-trifluorophenyl)-[1,2,4]-triazolo-[1,5-a]pyrimidine and 4-trifluoromethyl-piperidine as starting materials, the course of the process (a) according to the invention can be illustrated by the formula scheme below.

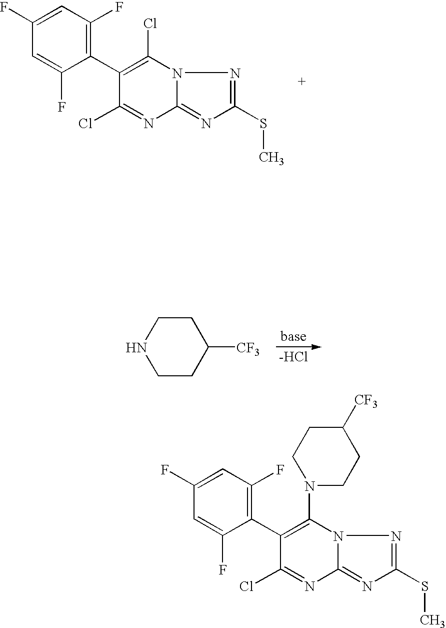

Using 5-chloro-2-(methylsulphanyl)-N-[(1S)-2,2,2-trifluoro-1-methyl-ethyl]-6-(2,4,6-trifluorophenyl)-[1,2,4]-triazolo-[1,5-a]pyrimidin-7-amine and dichloro-fluoromethane-sulphenyl chloride as starting materials, the course of the process (b) according to the invention can be illustrated by the formula scheme below.

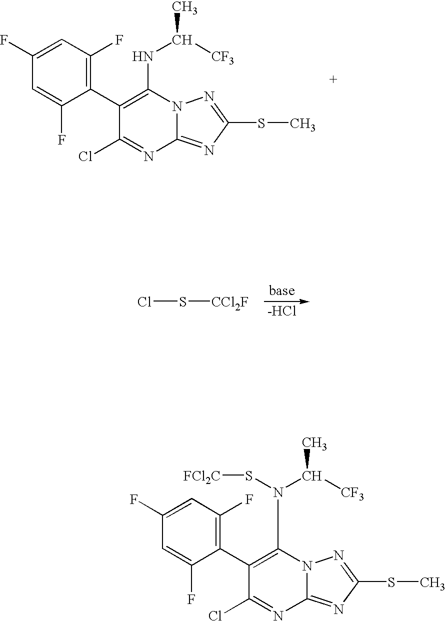

Using 5-chloro-2-(methyl-sulphanyl)-N-[(1S)-2,2,2-trifluoro-1-methyl-ethyl]-6-(2,4,6-trifluorophenyl)-[1,2,4]-triazolo[1,5-a]pyrimidin-7-amine as starting material and an excess of hydrogen peroxide as oxidizing agent, the course of the process (c) according to the invention can be illustrated by the formula scheme below:

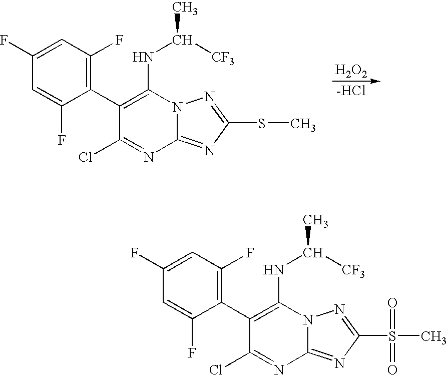

The formula (II) provides a general definition of the dihalogeno-triazolo-pyrimidines required as starting materials for carrying out the process (a) according to the invention. In this formula, R³, R⁴ and X preferably have those meanings which have already been mentioned in connection with the description of the substances of the formula (I) according to the invention as being preferred for these radicals. Y¹ preferably represents fluorine, chlorine or bromine, particularly preferably fluorine or chlorine.

The dihalogeno-triazolopyrimidines of the formula (II) are novel. These substances, too, are suitable for controlling unwanted microorganisms.

The dihalogeno-triazolopyrimidines can be prepared by

d) reacting dihydroxy-triazolo-pyrimidines of the formula

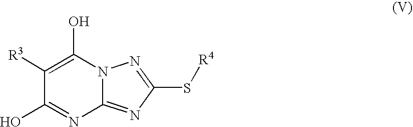

in which

R³ and R⁴ have the meanings given above,

with halogenating agents, if appropriate in the presence of a diluent.

The formula (V) provides a general definition of the dihydroxy-triazolopyrimidines required as starting materials for carrying out the process (d). In this formula, R³ and R⁴ preferably have those meanings which have already been mentioned in connection with the description of the substances of the formula (I) according to the invention as being preferred for these radicals.

The dihydroxytriazolopyrimidines of the formula (V), too, have hitherto not been known. They can be prepared by

e) reacting arylmalonic esters of the formula

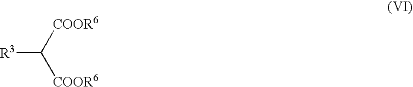

in which

R³ has the meanings given above and

R⁶ represents alkyl having 1 to 4 carbon atoms

with aminotriazoles of the formula

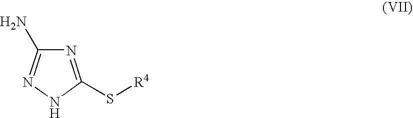

in which

R⁴ has the meanings given above,

if appropriate in the presence of a diluent and if appropriate in the presence of an acid binder.

The formula (VI) provides a general definition of the arylmalonic esters required as starting materials for carrying out the process (e). In this formula, R³ preferably has those meanings which have already been mentioned in connection with the description of the substances of the formula (I) according to the invention as being preferred for this radical. R⁶ preferably represents methyl or ethyl.

The arylmalonic esters of the formula (VI) are known or can be prepared by known methods (cf. U.S. Pat. No. 6,156,925).

The formula (VI) provides a general definition of the aminotriazoles furthermore required as starting materials for carrying out the process (e). In this formula, R⁴ preferably has those meanings which have already been mentioned in connection with the description of the substances of the formula (I) according to the invention as being preferred for this radical.

The aminotriazoles of the formula (VII) are known or can be prepared by known methods (cf. J. Heterocycl. Chem. (1982), 19(5), 1157-64).

Suitable diluents for carrying out the process (e) are all inert organic solvents which are customary for such reactions. Preference is given to using alcohols, such as methanol, ethanol, n-propanol, isopropanol, n-butanol and tert-butanol.

Suitable acid binders for carrying out the process (e) are all inorganic and organic bases which are customary for such reactions. Preference is given to using tertiary amines, such as tributylamine or pyridine. Amine used in excess may also act as diluent.

When carrying the process (e), the temperatures may be varied within a relatively wide range. In general, the process is carried out at temperatures between 20° C. and 200° C., preferably between 50° C. and 180° C.

The process (e) is generally carried out under atmospheric pressure. However, it is also possible to operate under elevated or reduced pressure.

When carrying out the process (e), arylmalonic esters of the formula (VI) and aminotriazole of the formula (VII) are generally employed in equimolar amounts.

However, it is also possible to use an excess of one or the other component. Work-up is carried out by customary methods.

Suitable halogenating agents for carrying out the process (d) are all components which are customarily used for replacing hydroxyl groups by halogen. Preference is given to using phosphorus trichloride, phosphorus tribromide, phosphorus pentachloride, phosphorus oxychloride, thionyl chloride, thionyl bromide or mixtures thereof. The corresponding fluoro compounds of the formula (II) can be prepared from the chloro or bromo compounds by reaction with potassium fluoride.

Suitable diluents for carrying out the process (d) are all solvents which are customary for such halogenations. Preference is given to using halogenated aliphatic or aromatic hydrocarbons, such as chlorobenzene. However, it is also possible for the halogenating agent itself, for example phosphorus oxychloride or a mixture of halogenating agents, to act as diluent.

When carrying out the process (d), too, the temperatures can be varied within a relatively wide range. In general, the process is carried out at temperatures between 0° C. and 150° C., preferably between 10° C. and 120° C.

The process (d) is generally carried out under atmospheric pressure. However, it is also possible to operate under elevated pressure.

When carrying out the process (d), the dihydroxy-triazolopyrimidine of the formula (V) is generally reacted with an excess of halogenating agent. Work-up is carried out by customary methods.

The formula (III) provides a general definition of the amines furthermore required as starting materials for carrying out the process (a) according to the invention. In this formula (III), R¹ and R² preferably have those meanings which have already been mentioned in connection with the description of the compounds of the formula (I) according to the invention as being preferred for R¹ and R².

The amines of the formula (M) are known in some cases.

Novel are the amines of the formula

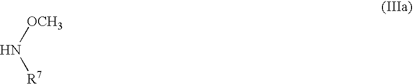

in which

R⁷ represents isobutyl, 2-methoxyethyl or represents

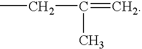

The amines of the formula (IIIa) can be prepared by

f) in a first stage reacting ethyl N-methoxycarbamate of the formula

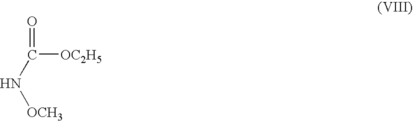

with halogen compounds of the formula

R⁷—X¹ (IX)

in which

R⁷ has the meanings given above and

X¹ represents bromine or iodine

in the presence of a base and in the presence of a diluent and in a second stage reacting the resulting carbamates of the formula

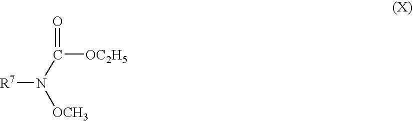

in which

R⁷ has the meanings given above

with potassium hydroxide in the presence of ethanol and water.

Also novel are amines of the formula

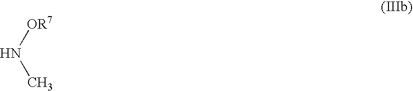

in which

R⁷ has the meanings given above.

The amines of the formula (IIIb) can be prepared by

g) in a first stage reacting ethyl N-hydroxy-N-methylcarbamate of the formula

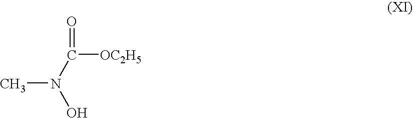

with halogen compounds of the formula

R⁷—X¹ (IX)

in which

R⁷ and X¹ have the meanings given above

in the presence of a base and in the presence of a diluent and in a second stage reacting the resulting carbamates of the formula

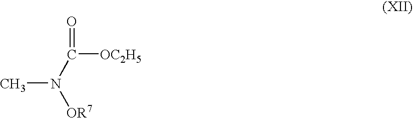

in which

R⁷ has the meanings given above

with potassium hydroxide in the presence of ethanol and water.

Also novel are trifluoroisopropylamines of the formula

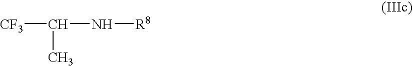

in which

R⁸ represents methyl, ethyl or propyl.

The trifluoroisopropylamines of the formula (IIIc) can be prepared by

h) in a first stage reacting ethyl N-trifluoroisopropylcarbamate of the formula

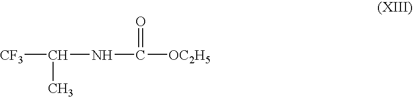

with halogen compounds of the formula

R⁸—X¹ (XIV)

in which

R⁸ and X¹ have the meanings given above

in the presence of a base and in the presence of a diluent and in a second stage reacting the resulting carbamates of the formula

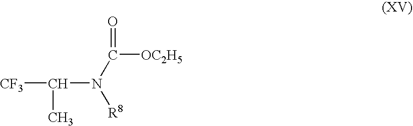

in which

R⁸ has the meanings given above

with potassium hydroxide in the presence of ethanol and water.

Also novel, finally, is 3-trifluoromethyl-3-amino-propene of the formula

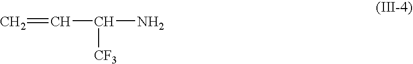

3-Trifluoromethyl-3-amino-propene of the Formula (IIId) can be Prepared by

(i) reacting the carbamate of the formula

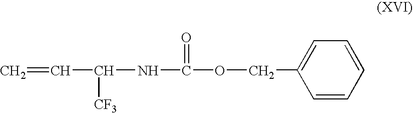

with aqueous hydrochloric acid.

The compounds of the formulae (VIII), (IX), (XI), (XIII), (XIV) and (XVI) required as starting materials for carrying out the processes (f)-(i) according to the inention are known or can be prepared by known methods.

Suitable acid acceptors when carrying out the first stage of the processes (f), (g) and (h) according to the invention include all organic and inorganic acid acceptors which are customary for such reactions.

With preference it is possible to use alkaline earth metal or alkali metal hydrides, hydroxides, amides, alkoxides, acetates, carbonates or bicarbonates, such as sodium hydride, sodium amide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate, for example, and also ammonium compounds, such as ammonium hydroxide, ammonium acetate and ammonium carbonate. As organic bases mention may be made of the following: tertiary amines, such as trimethylamine, triethylaamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Suitable diluents when carrying out the first stage of the processes (f), (g) and (h) according to the invention include in each case all customary inert organic solvents. With preference it is possible to use ethers, such as diethyl ether, dioxane, tetrahydrofuran, 1,2-dimethoxyethane, 1,2-diethoxyethane or anisole; amides, such as N,N-di-methylformamide, N,N-dimethylacetamide, N-methylformanilide or N-methylpyrrolidone; sulphones, such as sulpholane; or alcohols such as methanol, ethanol, isopropanol, tert-butanol or n-butanol.

The reaction temperatures when carrying out the first stage of the processes (f), (g) and (h) according to the invention can in each case be varied within a relatively wide range. It is normal to operate at temperatures between 0° C. and 150° C., preferably between 10° C. and 100° C.

When carrying out the first stage of the processes (f), (g) and (h) according to the invention it is normal to operate in each case under atmospheric pressure. It is, however, also possible to operate under increased pressure or, where no low-boiling components participate in the reaction, under reduced pressure.

When carrying out the first stage of the processes (f), (g) and (h) according to the invention

generally from 0.5 to 15 mol, preferably from 1 to 5 mol, of halogen compound of the formula (IX) are employed per mole of ethyl N-methoxycarbamate of the formula (VIII), or

generally from 0.5 to 15 mol, preferably from 1 to 5 mol, of halogen compound of the formula (IX) are employed per mole of ethyl N-hydroxy-N-methylcarbamate of the formula (XI), or

generally from 0.5 to 15 mol, preferably from 1 to 5 mol, of halogen compound of the formula (XV) are employed per mole of ethyl N-trifluoroisopropylcarbamate of the formula (XIII).

Working up takes place in each case in accordance with customary methods, for example by extraction and subsequent drying or by precipitation with subsequent filtration and drying. Any impurities still present can be removed in accordance with customary methods.

The compounds of the formulae (X), (XII) and (XV) obtained as intermediates when carrying out the first stage of the processes (f), (g) and (h) according to the invention are novel.

When carrying out the second stage of the processes (f), (g) and (h) according to the invention as well the reaction temperatures can in each case be varied within a relatively wide range. It is normal to operate at temperatures between 0° C. and 100° C., preferably between 10° C. and 80° C.

When carrying out the second stage of the processes (f), (g) and (h) according to the invention as well it is normal to operate in each case under atmospheric pressure. It is, however, again possible to operate in each case under increased pressure or, where the products to be isolated do not have a very low boiling point, under reduced pressure.

When carrying out the second stage of the processes (f), (g) and (h) according to the invention in each case an excess, preferably up to 10 mol, of potassium hydroxide is used per mole of a compound of the formula (X), (XII) or (XV). Working up takes place in accordance with customary methods. The amines are appropriately isolated in the form of their salts, generally by adding acid, preferably aqueous hydrochloric acid.

When carrying out the process (i) according to the invention the reaction temperatures can likewise be varied within a relatively wide range. It is normal to operate at temperatures between 10° C. and 150° C., preferably at reflux temperature.

When carrying out the process (i) according to the invention it is normal to operate under atmospheric pressure. It is, however, also possible to operate under increased pressure.

When carrying out the process (i) according to the invention an excess, preferably up to 10 mol, of aqueous hydrochloric acid is used per mole of carbamate of the formula (XVI). Working up takes place again in accordance with customary methods.

The formula (Ia) provides a general definition of the triazolopyrimidines required as starting materials for carrying out the process (b) according to the invention. In this formula, R², R³, R⁴ and X preferably have those meanings which have already been mentioned in connection with the description of the substances of the formula (I) according to the invention as being preferred for these radicals.

The triazolopyrimidines of the formula (Ia) are substances according to the invention. They can be prepared by process (a) according to the invention.

The formula (IV) provides a general definition of the sulphenyl halides furthermore required as starting materials for carrying out the process (b) according to the invention. In this formula, R⁵ preferably has those meanings which have already been mentioned in connection with the description of the compounds of the formula (I) according to the invention as being preferred for this radical.

Y² preferably represents fluorine, chlorine or bromine, particularly preferably chlorine.

The sulphenyl halides of the formula (IV) are known or can be prepared by known methods.

The formula (Ib) provides a general definition of the triazolopyrimidines required as starting materials for carrying out the process (c) according to the invention. In this formula, R¹, R², R³, R⁴ and X preferably have those meanings which have already been mentioned in connection with the description of the substances of the formula (I) according to the invention as being preferred for these radicals.

The triazolopyrimidines of the formula (Ib) are substances according to the invention. They can be prepared by process (a) or (b) according to the invention.

Suitable oxidizing agents for carrying out the process (c) according to the invention are all customary substances suitable for releasing oxygen. Preference is given to using hydrogen peroxide, its salts and its adducts with, for example, urea, and furthermore to tert-butyl peroxide, peracids or salts thereof, such as performic acid, peracetic acid, perpropionic acid, perbenzoic acid, 3-chloroperbenzoic acid, potassium hydrogen persulphate, potassium peroxodisulphate, sodium perborate, sodium percarbonate or sodium peroxodisuluphate, and furthermore also to potassium permanganate or sodium perrhenate, and additionally also to chlorous or hypochlorous acid or solutions of their metal salts in water.

Suitable diluents for carrying out the process (a) according to the invention are all customary inert organic solvents. Preference is given to using aliphatic, alicyclic or aromatic hydrocarbons, such as hexane, heptane, cyclohexane or toluene; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl tert-butyl ether, dioxane, tetrahydrofuran, 1,2-dimethoxyethane or 1,2-diethoxyethane; amides, such as N,N-dimethylformamide, N,N-dimethylacetamide or N-methylpyrrolidone; esters, such as methyl acetate or ethyl acetate; sulphoxides, such as dimethyl sulphoxide; sulphones, such as sulpholane.

Suitable acid acceptors for carrying out the process (a) according to the invention are all acid binders which are customary for such reactions. Preference is given to using tertiary amines, such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process (a) according to the invention, the reaction temperatures may be varied within a relatively wide range. In general, the process is carried out at temperatures between 0° C. and 150° C., preferably at temperatures between 0° C. and 80° C.

Both the process (a) and the process (b) according to the invention are generally carried out under atmospheric pressure. However, it is also possible to operate under elevated or reduced pressure, in general between 0.1 and 10 bar.

When carrying out the process (a) according to the invention, in general from 0.5 to 10 mol, preferably from 0.8 to 2 mol, of the amine of the formula (III) are employed per mole of dihalogeno-triazolo-pyriridine of the formula (II). Work-up is carried out by customary methods.

Suitable diluents for carrying out the process (b) according to the invention are all customary inert organic solvents. Preference is given to using ethers, such as diethyl ether, dioxane, tetrahydrofuran, 1,2-dimethoxyethane, 1,2-diethoxyethane or anisole; amides, such as N,N-dimethylformamide, N,N-dimethylacetamide, N-methylformanilide or N-methylpyrrolidone; sulphones, such as sulpholane.

Suitable acid acceptors for carrying out the process (b) according to the invention are all inorganic and organic bases which are customary for such reactions. Preference is given to using alkaline earth metal or alkali metal hydrides, hydroxides, amides, alkoxides, acetates, carbonates or bicarbonates, such as, for example, sodium hydride, sodium amide, sodium methoxide, sodium ethoxide, potassium tert-butoxide, sodium hydroxide, potassium hydroxide, sodium acetate, potassium acetate, calcium acetate, sodium carbonate, potassium carbonate, potassium bicarbonate and sodium bicarbonate, and furthermore ammonium compounds, such as ammonium hydroxide, ammonium acetate and ammonium carbonate.

When carrying out the process (b) according to the invention, the reaction temperatures can also be varied within a relatively wide range. In general, the process is carried out at temperatures between −40° C. and +120° C., preferably between −20° C. and +50° C.

When carrying out the process (b) according to the invention, in general from 1 to 15 mol, preferably from 1 to 8 mol, of sulphenyl halide of the formula (IV) are employed per mole of triazolopyrimidine of the formula (Ia). Work-up is carried out by customary methods.

Suitable diluents for carrying out the process (c) according to the invention are all inert organic solvents which are customary for such reactions. With preference it is possible to use aliphatic, alicyclic or aromatic hydrocarbons, such as petroleum ether, hexane, heptane, cyclohexane, methylcyclohexane, benzene, toluene, xylene or decaline; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; ketones, such as acetone, butanone, methyl isobutyl ketone or cyclohexanone; acids, such as formic acid, acetic acid or propionic acid, nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; sulphones, such as sulpholane; alcohols, such as methanol, ethanol, n- or isopropanol, n-, iso-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

Suitable catalysts for carrying out the process (c) according to the invention are all reaction promoters which are customary for such reactions. Preference is given to using salts or metals of the IV., V. and VI. transition group of the Periodic Table of the Elements. Examples which may be mentioned are sodium (meta)vanadate, sodium molybdate and sodium tungstate.

In a particular variant, the process (c) according to the invention can also be carried out in a two-phase system, such as, for example, water/toluene or water/dichloromethane, if appropriate in the presence of a suitable phase-transfer catalyst. Examples of such catalysts which may be mentioned are: tetrabutylammonium iodide, tetrabutylammonium bromide, tetrabutylammonium chloride, tributyl-methylphosphonium bromide, trimethyl-C₁₃/C₁₅-alkylammonium chloride, trimethyl-C₁₃/C₁₅-alkylammonium bromide, dibenzyl-dimethyl-ammoniummethylsulphate, dimethyl-C₁₂/C₁₄-alkyl-benzylammonium chloride, dimethyl-C₁₂/C₁₄-alkyl-benzylammonium bromide, tetrabutylammonium hydroxide, triethylbenzylammonium chloride, methyltrioctylammonium chloride, trimethylbenzylammonium chloride, 15-crown-5, 18-crown-6 or tris-[2-(2-methoxyethoxy)-ethyl]-amine.

When carrying out the process (c) according to the invention, the reaction temperatures may also be varied within a relatively wide range. In general, the process is carried out at temperatures between 0° C. and 150° C., preferably between 0° C. and 80° C.

The process (c) according to the invention is also generally carried out under atmospheric pressure. However, it is also possible to operate under elevated pressure.

If the process (c) according to the invention is carried out with the intention to prepare triazolopyrimidines of the formula (I) in which n represents 1, in general from 1 to 1.5 equivalents, preferably from 1 to 1.2 equivalents, of oxidizing agent are employed per mole of triazolopyrimidine of the formula (Ib).

If the process (c) according to the invention is carried out with the intention to prepare triazolopyrimidines of the formula (I) in which n represents 2, in general from 2 to 10 equivalents, preferably from 2 to 5 equivalents, of oxidizing agent are employed per mole of triazolopyrimidine of the formula (Ib). Work-up is in each case carried out by customary methods.

Acids suitable for preparing acid addition salts of triazolopyrimidines of the formula (I) are preferably those acids already stated as being preferred acids in connection with the description of the acid addition salts according to the invention.

The acid addition salts of the compounds of the formula (I) can be obtained in a simple way by customary salt-forming methods, e.g. by dissolving a compound of the formula (I) in a suitably inert solvent and adding the acid, e.g. hydrochloric acid, and can be isolated in a known way, e.g. by filtration, and can optionally be purified by washing with an inert organic solvent.

The substances according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae. Some pathogens causing fungal and bacterial diseases which come under the generic names listed above may be mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea (conidia form: Drechslera, syn: Helminthosporium);

Cochliobolus species, such as, for example, Cochliobolus sativus (conidia form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Alternaria species, such as, for example, Alternaria brassicae; and

Pseudocercosporella species, such as, for example, Pseudocercosporella herpotrichoides.

The active compounds according to the invention also have very good fortifying action in plants. Accordingly, they can be used for mobilizing the defences of the plant against attack by unwanted microorganisms.

In the present context, plant-fortifying (resistance-inducing) substances are to be understood as meaning those substances which are capable of stimulating the defence system of plants such that, when the treated plants are subsequently inoculated with unwanted microorganisms, they show substantial resistance against these microorganisms.

In the present case, unwanted microorganisms are to be understood as meaning phytopathogenic fungi, bacteria and viruses. Accordingly, the substances according to the invention can be used to protect plants for a certain period after the treatment against attack by the pathogens mentioned. The period for which protection is provided generally extends over 1 to 10 days, preferably 1 to 7 days, after the treatment of the plants with the active compounds.

The fact that the active compounds are well tolerated by plants at the concentrations required for controlling plant-diseases permits the treatment of above-ground parts of plants, of propagation stock and seeds, and of the soil.

The active compounds according to the invention can be employed particularly successfully for controlling diseases in Viticulture and fruit and vegetable growing such as, for example, against Venturia or Podosphaera species.

The active compounds according to the invention are also suitable for increasing the yield of crops. In addition, they show reduced toxicity and are well tolerated by plants.

At certain concentrations and application rates, the active compounds according to the invention can also be used as herbicides, for influencing plant growth and for controlling animal pests. If appropriate, they can also be used as intermediates and precursors for the synthesis of further active compounds.

The active compounds according to the invention can be used to treat all plants and parts of plants. By plants are understood here all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant varieties which can or cannot be protected by varietal property rights. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment of the plants and the parts of plants with the active compounds according to the invention is carried out directly or by action on their surroundings, habitat or storage space, according to customary treatment methods, for example by dipping, spraying, evaporating, atomizing, broadcasting, spreading-on and, in the case of propagation material, in particular in the case of seeds, furthermore by one- or multi-layer coating.

In the protection of materials, the compounds according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes), and against slime organisms and algae.

Microorganisms of the following genera may be mentioned as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa, and

Staphylococcus, such as Staphylococcus aureus.

Depending on their particular physical and/or chemical properties, the active compounds can be converted to the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents.

Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided, silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 per cent by weight of active compound, preferably between 0.5 and 90%.

The active compounds according to the invention can be used as such or in their formulations, also in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Examples of suitable mixing components are the following:

Fungicides:

aldimorph, ampropylfos, ampropylfos potassium, andoprim, anilazine, azaconazole, azoxystrobin,

benalaxyl, benodanil, benomyl, benzamacril, benzamacril-isobutyl, bialaphos, binapacryl, biphenyl, bitertanol, blasticidin-S, bromuconazole, bupirimate, buthiobate,

calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, carpropamide,

debacarb, dichlorophen, diclobutrazole, diclofluanid, diclomezine, dicloran, diethofencarb, difenoconazole, dimethirimol, dimethomorph, diniconazole,

diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoroimide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, fenhexamide,

guazatine,

hexachlorobenzene, hexaconazole, hymexazole,

imazalil, imibenconazole, iminoctadine, iminoctadine albesilate, iminoctadine triacetate, iodocarb, ipconazole, iprobenfos (IBP), iprodione, irumamycin, isoprothiolane, isovaledione, iprovalicarb

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur,

quinconazole, quintozene (PCNB), quinoxyfen,

sulphur and sulphur preparations, spiroxamines,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole,

uniconazole,

validamycin A, vinclozolin, viniconazole,

zarilamide, zineb, ziram and also

Dagger G,

OK-8705,

OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-β-methoxy-α-methoyl-1H-1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide,

1-(2,4-dichlorophenyl)-2-(1H-1,2,4-triazol-1-yl)-ethanone O-(phenylmethyl)-oxime,

1-(2-methyl-1-naphthalenyl)-1H-pyrrole-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinole,

2′,6′-dibromo-2-methyl-4′-trifluoromethoxy-4′-trifluoro-methyl-1,3-thiazole-5-carboxanilide,

2,6-dichloro-5-(methylthio)-4-pyrimidinyl-thiocyanate,

2,6-dichloro-N-(4-triifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-β-D-glycopyranosyl)-α-D-glucopyranosyl]-amino]-4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)-acetamide,

2-phenylphenol (OPP),

3,4-dichloro-1-[4-(difluoromethoxy)-phenyl]-1H-pyrrole-2,5-dione,

3,5-dichloro-N-[cyano[(1-methyl-2-propynyl)-oxy]-methyl]-benzamide,

3-(1,1-dimethylpropyl-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-a]quinazolin-5(4H)-one,

8-hydroxyquinoline sulphate,

9H-xanthene-2-[(phenylamino)-carbonyl]-9-carboxylic hydrazide,

bis-(1-methylethyl)-3-methyl-4-[(3-methylbenzoyl)-oxy]-2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl-2-methylpropyl]-2,6-dimethyl-morpholine-hydrochloride,

ethyl [(4-chlorophenyl)-azo]-cyanoacetate,

potassium hydrogen carbonate,

methanetetrathiol sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)-4-methyl-3-nitro-benzenesulphonamide,

N-(4-cyclohexylphenyl)-1,4,5,6-tetrahydro-2-pyrinidineamine,

N-(4-hexylphenyl)-1,4,5,6-tetrahydro-2-pyrimidineamine,

N-(5-chloro-2-methylphenyl)-2-methoxy-N-(2-oxo-3-oxazolidinyl)-acetamide,

N-(6-methoxy-3-pyridinyl)-cyclopropanecarboxamide,

N-[2,2,2-trichloro-1-[(chloroacetyl)-amino]-ethyl]-benzamide,

N-[3-chloro-4,5-bis-(2-propinyloxy)-phenyl]-N′-methoxy-methanimidamide,

N-formyl-N-hydroxy-DL-alanine-sodium salt,

O,O-diethyl[2-(dipropylamino)-2-oxoethyl]-ethylphosphoramidothioate,

O-methyl S-phenyl phenylpropylphosphoraridothioate,

S-methyl 1,2,3-benzothiadiazole-7-carbothioate,

spiro[2H]-1-benzopyrane-2,1′(3′H)-isobenzofuran]-3′-one,

4-[(3,4-dimethoxyphenyl)-3-(4-fluorophenyl)-acryloyl]-morpholine.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

abamectin, acephate, acetamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, dicofol, diflubenzuron, dimethoat, dimethylvinphos, diofenolan, disulfoton, docusat-sodium, dofenapyn,

eflusilanate, emamectin, empenthrin, endosulfan, Entomopfthora spp., esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim,. fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, indoxacarb, isazofos, isofenphos, isoxathion, ivermectin,

nuclear polyhedrosis viruses,

lambda-cyhalothrin, lufenuron,

malathion, mecarbam, metaldehyde, methamidophos, Metharhiziuni anisopliae, Metharhizium flavoviride, methidathion, methiocarb, methoprene, methomyl, methoxyfenozide, metolcarb, metoxadiazone, mevinphos, milbemectin, milbemycin, monocrotophos,

naled, nitenpyram, nithiazine, novaluron,

omethoate, oxamyl, oxydemethon M,

Paecilomyces fumosoroseus, parathion A, parathion M, permethrin, phenthoate, phorat, phosalone, phosmet, phosphamidon, phoxim, pirimicarb, pirimiphos A, pirimiphos M, profenofos, promecarb, propargite, propoxur, prothiofos, prothoat, pymetrozine, pyraclofos, pyresmethrin, pyrethrum, pyridaben, pyridathion, pyrimidifen, pyriproxyfen,

quinalphos,

ribavirin,

salithion, sebufos, silafluofen, spinosad, spirodiclofen, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, tetradifon theta-cypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,

vamidothion, vaniliprole, Verticillium lecanii,

YI 5302

zeta-cypermethrin, zolaprofos

(1R-cis)-[5-(phenylmethyl)-3-furanyl]-methyl-3-[(dihydro-2-oxo-3(2H)-furanylidene)-methyl]-2,2-dimethylcyclopropanecarboxylate,

(3-phenoxyphenyl)-methyl-2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)-4-[4-(1,1-dimethylethyl)phenyl]-4,5-dihydro-oxazole,

2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl[2-(dipropylamino)-2-oxoethyl]-ethylphosphoramidothioate,

N-cyanomethyl-4-trifluoromethyl-nicotinamide,

3,5-dichloro-1-(3,3-dichloro-2-propenyloxy)-4-[3-(5-trifluoromethylpyridine-2-yloxy)-propoxy]-benzene.

A mixture with other known active compounds, such as herbicides, or with fertilizers and growth regulators, is also possible.

In addition, the compounds of the formula (I) according to the invention also have very good antimycotic activity. They have a very broad antimycotic activity spectrum in particular against dermatophytes and yeasts, moulds and diphasic fungi, (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The list of these fungi by no means limits the mycotic spectrum covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10,000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10,000 g/ha, preferably between 1 and 5000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetical engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or proccessability of the harvested products are possible which exceed the effects which were actually to be expected.

The transgenic plants or plant cultivars (i.e. those obtained by genetical engineering) which are preferably treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or proccessability of the harvested products. Further and particularly emphasised examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasised are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasised are the increased defence of the plants to fungi, bacteria and viruses by systemic acquired resistance (SAR), systemin, phytoalexins, elicitors and resistance genes and correspondingly expressed proteins and toxins. Traits that are furthermore particularly emphasised are the increased tolerance of the plants to certain herbicidally active compounds, for example imidazolinones, sulphonylureas, glyphosate or phosphinotricin (for example the “PAT”gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylurea, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plants will be developed and/or marketed in the future.

The plants listed can be treated according to the invention in a particularly advantageous manner with the compounds of the general formula (I) according to the invention. The preferred ranges stated above for the active compounds also apply to the treatment of these plants. Particular emphasis is given to the treatment of plants with the compounds specifically mentioned in the present text.

The invention is illustrated by the examples below.

### PREPARATION EXAMPLES

### Example 1

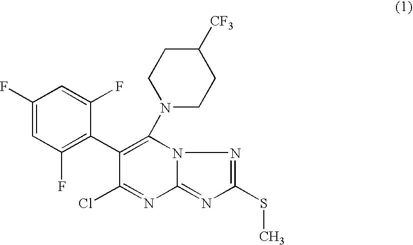

Process (a)

0.277 g of triethylamine is added to a solution of 1 g (2.378 mmol) of 5,7-dichloro-2-(methylsulphanyl)-6-(2,4,6-trifluorophenyl)[1,2,4]triazolo[1,5-a]pyrimidine and 0.419 g (2.378 mmol) of 4-trifluoromethy]piperidine in 20 ml of dichloromethane. The mixture is stirred at room temperature for 18 hours. With stirring, 1N hydrochloric acid is then added to the reaction mixture until the pH of the mixture is 1-2 (about 50 ml). The organic phase is separated off, dried over sodium sulphate and concentrated under reduced pressure. The residue is triturated with diisopropyl ether and filtered off with suction. This gives 1.1 g (83.4% of theory) of 5-chloro-2-(methylsulphanyl)-7-[4-(trifluoromethyl)-1-piperidinyl]-6-(2,4,6-trifluorophenyl)-[1,2,4]triazolo[1,5-a]pyrimidine.

HPLC: logP=4.10

### Example 2

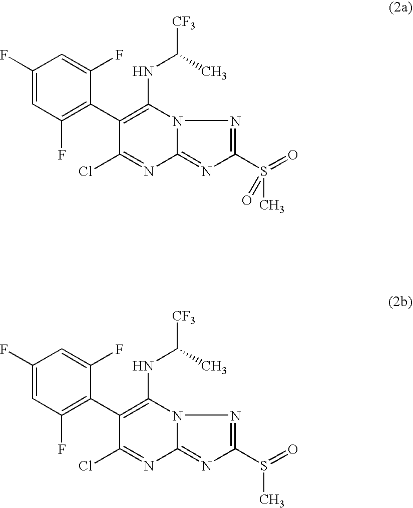

Process (c)

0.1 g of ammonium molybdate and 0.17 ml of 98 to 100% strength formic acid are added to a solution of 1 g (2.264 mmol) of 5-chloro-2-(methylsulphanyl)-N-[(1S)-2,2,2-trifluoro-1-methylethyl]-6-(2,4,6-trifluorophenyl)[1,2,4]triazolo[1,5-a]-pyrimidin-7-amine in 20 ml of dichloromethane. 0.59 ml (6.8 mmol) of 35% strength hydrogen peroxide is gradually added dropwise, and the mixture is stirred for 16 hours. 50 ml of water are then added to the reaction mixture. The organic phase is then separated off, washed with 50 ml of 10% strength aqueous sodium hydrogen sulphite solution, dried over sodium sulphate and concentrated under reduced pressure. The residue is triturated with diisopropyl ether and filtered off with suction. The crude product is chromatographed on silica gel, initially using n-hexane/ethyl acetate (1:1) and finally pure ethyl acetate. This gives 260 mg (24% of theory) of 5-chloro-2-(methylsulphonyl)-N-[(1S)-2,2,2-trifluoro-1-methylethyl]-6-(2,4,6-tri-fluorophenyl)[1,2,4]triazolo[1,5-a]pyrimidin-7-amine (2a) and 360 mg (34% of theory) of 5-chloro-2-(methylsulphinyl)-N-[(1S)-2,2,2-trifluoro-1-methylethyl]-6-(2,4,6-trifluorophenyl)[1,2,4]triazolo[1,5-a]pyrimidin-7-amine (2b).

2a: HPLC: logP=2.94

2b: HPLC: logP=2.50

The compounds of the formula (I) listed in Table 1 below are also obtained by the methods given above.

TABLE 1
(I)

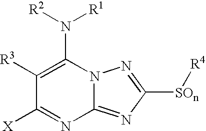

| Ex. | R¹ | | | | | | logP | m.p.: |
|---|---|---|---|---|---|---|---|---|
| No. | (***) | R² | R³ | R⁴ | n | X | (**) | (° C.) |
| 3 | Cyclopentyl | —H | 2-Chlorophenyl | —CH₃ | 0 | Cl | | 119- |
| | | | | | | | | 122 |
| 4 | Cyclopentyl | —H | 2,6-Dichlorophenyl | —CH₃ | 0 | Cl | 4.17 | |
| 5 | —CH₂—CH₂—CH(CH₃)—CH₂—CH₂— | * | 2,6-Dichlorophenyl | —CH₃ | 0 | Cl | 4.9 | |
| 6 | Cyclopentyl | —H | 2,6-Dichlorophenyl | —CH₃ | 2 | Cl | 3.35 | |
| 7 | —CH₂—CH₂—CH(CH₃)—CH₂—CH₂— | * | 2,6-Dichlorophenyl | —CH₃ | 2 | Cl | 3.73 | |
| 8 | 2,2,2-Trifluoro-1-methylethyl | —H | 2,6-Dichlorophenyl | —CH₃ | 0 | Cl | 3.82 | |
| 9 | 2,2,2-Trifluoro-1-methylethyl | —H | 2,4,6-Trifluorophenyl | —CH₃ | 0 | Cl | 3.58 | |
| 10 | —CH₂—CH₂—CF₃ | —H | 2,4,6-Trifluorophenyl | —CH₃ | 0 | Cl | 3.36 | |
| 11 | —C₂H₅ | —C₂H₅ | 2,4,6-Trifluorophenyl | —CH₃ | 0 | Cl | 3.89 | |
| 12 | —CH₂—CF₃ | —H | 2,4,6-Trifluorophenyl | —CH₃ | 0 | Cl | 3.23 | |
| 13 | N-Morpholinyl | —H | 2,4,6-Trifluorophenyl | —CH₃ | 0 | Cl | 2.72 | 202-03 |
| 14 | —NH—CH₂—CF₃ | —H | 2,4,6-Trifluorophenyl | —CH₃ | 0 | Cl | 3.07 | 157-59° |
| 15 | —CH₂—CH₂—O—CH₂—CH₂— | * | 2,4,6-Trifluorophenyl | —CH₃ | 0 | Cl | 2.99 | 208-09 |
| 16 | 1-Piperidinyl | —H | 2,4,6-Trifluorophenyl | —CH₃ | 0 | Cl | 3.81 | 209-11 |
| 17 | —CH₂—CH₂—CH₂—CH₂—CH₂— | * | 2,4,6-Trifluorophenyl | —CH₃ | 0 | Cl | 4.09 | |
| 18 | —NH—CH₂—CF₃ | —H | 2-Chlorophenyl | —CH₃ | 0 | Cl | 3.1 | |
| 19 | Ethylamino | —H | 2,4,6-Trifluorophenyl | —CH₃ | 0 | Cl | 2.93 | 215-18 |
| 20 | 1-Cyclopropylethylamino | —H | 2,4,6-Trifluorophenyl | —CH₃ | 0 | Cl | 3.74 | |
| 21 | —CH₂—C(CH₃)═CH₂ | —C₂H₅ | 2,4,6-Trifluorophenyl | —CH₃ | 0 | Cl | 4.43 | |
| 22 | -i-Propyl | —H | 2,4,6-Trifluorophenyl | —CH₃ | 0 | Cl | 3.36 | |
| 23 | —NH—CH₃ | —CH₃ | 2,4,6-Trifluorophenyl | —CH₃ | 0 | Cl | 3.08 | 213-14 |
| 24 | -i-Propyl | —H | 2-Chlorophenyl | —CH₃ | 0 | Cl | 3.43 | |
| 25 | n-Propyl | —H | 2-Chlorophenyl | —CH₃ | 0 | Cl | 3.4 | |
| 26 | —CH₂—CH₂—CH₂—CH₂— | * | 2-Chlorophenyl | —CH₃ | 0 | Cl | 3.53 | |
| 27 | —CH₂—CH₂—CH₂—CH₂—CH₂— | * | 2-Chlorophenyl | —CH₃ | 0 | Cl | 4.21 | |
| 28 | —CH₂—CH₂—O—CH₂—CH₂— | * | 2-Chlorophenyl | —CH₃ | 0 | Cl | 2.98 | |
| 29 | t-Butylamino | —H | 2,4,6-Trifluorophenyl | —CH₃ | 0 | Cl | 3.66 | 216-17 |
| 30 | n-Butylamino | —H | 2,4,6-Trifluorophenyl | —CH₃ | 0 | Cl | 3.63 | 227-28 |
| 31 | —NH—CH₂—CH(CH₃)₂ | —H | 2,4,6-Trifluorophenyl | —CH₃ | 0 | Cl | 3.66 | 226-28 |
| 32 | 2-Hydroxyethylamino | —H | 2,4,6-Trifluorophenyl | —CH₃ | 0 | Cl | 2.14 | |
| 33 | -i-Propyl | —H | 2-Chloro-6-fluorophenyl | —CH₃ | 0 | Cl | 3.42 | |
| 34 | —CH₂—CH(CH₃)—O—CH(CH₃)—CH₂— | * | 2-Chlorophenyl | —CH₃ | 0 | Cl | 3.79 | |
| 35 | 2,2,2-Trifluoro-1-methylethyl | —H | 2-Chlorophenyl | —CH₃ | 0 | Cl | 3.63 | |
| 36 | —N═CH—N═CH— | * | 2-Chlorophenyl | —CH₃ | 0 | Cl | 2.73 | |
| 37 | 2,2,2-Trifluoro-1-methylethyl | —H | 2-Chlorophenyl | —CH₃ | 0 | Cl | 3.64 | |
| 38 | 2,2,2-Trifluoro-1-methylethyl | —H | 2-Chloro-6-fluorophenyl | —CH₃ | 0 | Cl | 3.54 | |
| 39 | —CH₂—C(CH₃)═CH₂ | —H | 2,4,6-Trifluorophenyl | —CH₃ | 0 | Cl | 3.34 | |
| 40 | —CH₂—CN | —H | 2,4,6-Trifluorophenyl | —CH₃ | 0 | Cl | 2.48 | |
| 41 | -i-Propyl | —H | 2,6-Dichloro-3-fluoro-5- | —CH₃ | 0 | Cl | 4.26 | 173-75 |
| | | | trifluoromethylphenyl | | | | | |
| 42 | Cyclohexylmethylamino | —H | 2,4,6-Trifluorophenyl | —CH₃ | 0 | Cl | 4.41 | 178-81 |
| 43 | 2,2,2-Trifluoro-1-methylethyl | —H | 2,6-Dichloro-3-fluoro-5- | —CH₃ | 0 | Cl | 4.46 | 168-70 |
| | | | trifluoromethylphenyl | | | | | |
| 44 | i-Propylamino | —H | 2,6-Dichloro-3-fluoro-5- | —CH₃ | 0 | Cl | 4.24 | 175-80 |
| | | | trifluoromethylphenyl | | | | | |
| 45 | —CH₂—CN | —H | Pentafluorophenyl | —CH₃ | 0 | Cl | 2.89 | |
| 46 | —CH₂—CH₂—CH₂—CH₂—CH₂— | * | Pentafluorophenyl | —CH₃ | 0 | Cl | 4.43 | |
| 47 | n-Propyl | —H | Pentafluorophenyl | —CH₃ | 0 | Cl | 3.78 | |
| 48 | Cyclopentyl | —H | Pentafluorophenyl | —CH₃ | 0 | Cl | 4.28 | |
| 49 | —C₂H₅ | —C₂H₅ | Pentafluorophenyl | —CH₃ | 0 | Cl | 4.25 | |
| 50 | Cyclopropyl | —H | Pentafluorophenyl | —CH₃ | 0 | Cl | 3.52 | |
| 51 | Cyclopropylmethyl | —H | Pentafluorophenyl | —CH₃ | 0 | Cl | 3.86 | |
| 52 | -i-Propyl | —H | Pentafluorophenyl | —CH₃ | 0 | Cl | 3.81 | |
| 53 | —CH₂—CF₃ | —H | Pentafluorophenyl | —CH₃ | 0 | Cl | 3.62 | |
| 54 | —CH₂—CH₂—CH(CF₃)—CH₂—CH₂— | * | Pentafluorophenyl | —CH₃ | 0 | Cl | 4.42 | |
| 55 | 2,2,2-Trifluoro-1-methylethyl | —H | Pentafluorophenyl | —CH₃ | 0 | Cl | 3.97 | |
| 56 | N-Morpholinyl | —H | Pentafluorophenyl | —CH₃ | 0 | Cl | 3.22 | |
| 57 | Dimethylamino | —H | Pentafluorophenyl | —CH₃ | 0 | Cl | 3.57 | |
| 58 | N-Morpholinyl | —H | 2,4,6-Trifluorophenyl | —CH₃ | 1 | Cl | 1.99 | |
| 59 | N-Morpholinyl | —H | 2,4,6-Trifluorophenyl | —CH₃ | 2 | Cl | 2.37 | 140-42 dec. |
| 60 | —O—CH₃ | —CH₃ | 2-Chlorophenyl | —CH₃ | 0 | Cl | 3.37 | |
| 61 | —N═CH₂— | —CH₃ | 2,4,6-Trifluorophenyl | —CH₃ | 2 | Cl | 2.61 | 185-88 |
| 62 | 2-Methoxyethyl | —C₂H₅ | 2,4,6-Trifluorophenyl | —CH₃ | 0 | Cl | 3.61 | |
| 63 | —N═CH₂— | —CH₃ | 2,4,6-Trifluorophenyl | —CH₃ | 1 | Cl | 2.14 | |
| 64 | —CH₂—CH₂—N(CH₃)—CH₂—CH₂— | * | 2,4,6-Trifluorophenyl | —CH₃ | 0 | Cl | 1.47 | |
| 65 | —CH₂—CH₂—CH₂—CH₂— | * | 2,4,6-Trifluorophenyl | —CH₃ | 0 | Cl | 3.45 | |
| 66 | n-Propyl | —H | 2,4,6-Trifluorophenyl | —CH₃ | 0 | Cl | 3.34 | |
| 67 | Cyclopentyl | —H | 2,4,6-Trifluorophenyl | —CH₃ | 0 | Cl | 3.87 | |
| 68 | 2-Methoxyethyl | —H | 2,4,6-Trifluorophenyl | —CH₃ | 0 | Cl | 2.88 | |
| 69 | Cyclopropyl | —H | 2,4,6-Trifluorophenyl | —CH₃ | 0 | Cl | 3.11 | |
| 70 | —CH₂—CH₂—S—CH₂—CH₂— | * | 2,4,6-Trifluorophenyl | —CH₃ | 6 | Cl | 3.66 | |
| 71 | Cyclopropylmethyl | —H | 2,4,6-Trifluorophenyl | —CH₃ | 0 | Cl | 3.44 | |
| 72 | —CH₃ | —CH₃ | 2-Chlorophenyl | —CH₃ | 0 | Cl | | |
| 73 | —C₂H₅ | —H | 2-Chlorophenyl | —CH₃ | 0 | Cl | | |
| 74 | —C₂H₅ | —C₂H₅ | 2-Chlorophenyl | —CH₃ | 0 | Cl | | |
| 75 | 2-Methoxyethyl | —H | 2-Chlorophenyl | —CH₃ | 0 | Cl | | |
| 76 | —CH₃ | —H | 2-Chlorophenyl | —CH₃ | 0 | Cl | | |
| 77 | Cyclopropylmethyl | —H | 2-Chlorophenyl | —CH₃ | 0 | Cl | | |
| 78 | —CH₂—CH₂—S—CH₂—CH₂— | * | 2-Chlorophenyl | —CH₃ | 0 | Cl | | |
| 79 | —CH₂—CH₂—CH(CF₃)—CH₂—CH₂— | * | 2-Chlorophenyl | —CH₃ | 0 | Cl | | |
| 80 | Pyrrolidin-1-yl | —H | 2-Chlorophenyl | —CH₃ | 0 | Cl | | |
| 81 | Cyclohexyl | —H | 2-Chlorophenyl | —CH₃ | 0 | Cl | | |
| 82 | 1-Cyclohexylethyl | —H | 2-Chlorophenyl | —CH₃ | 0 | Cl | | |
| 83 | —CH₂—CF₃ | —H | 2-Chlorophenyl | —CH₃ | 0 | Cl | | |
| 84 | 1-Butoxy | —H | 2,4,6-Trifluorophenyl | —CH₃ | 0 | Cl | 1.83 | |
| 85 | —O—C₂H₅ | —H | 2,4,6-Trifluorophenyl | —CH₃ | 0 | Cl | 2.99 | |
| 86 | 4-Trifluoromethylbenzyloxy | —H | 2,4,6-Trifluorophenyl | —CH₃ | 0 | Cl | 4.06 | |
| 87 | —O—CH(CH₃)—CH₂—CH₃ | —H | 2,4,6-Trifluorophenyl | —CH₃ | 0 | Cl | 3.6 | |
| 88 | Allyloxy | —H | 2,4,6-Trifluorophenyl | —CH₃ | 0 | Cl | 3.15 | |
| 89 | t-Butoxy | —H | 2,4,6-Trifluorophenyl | —CH₃ | 0 | Cl | 3.52 | |
| 90 | —O—CH₃ | —H | 2,4,6-Trifluorophenyl | —CH₃ | 0 | Cl | 2.74 | |
| 91 | 2,2,2-Trifluoro-1-methylethyl | —H | 3-Chlor-4-fluorophenyl | —CH₃ | 0 | Cl | 3.93 | |
| 92 | -i-Propyl | —H | 3-Chlor-4-fluorophenyl | —CH₃ | 0 | Cl | 3.66 | |
| 93 | 2,2,2-Trifluoro-1-methylethyl | —H | 2-Chloro-6-fluorophenyl | —CH₃ | 0 | Cl | 3.63 | |
| 94 | —CH₂—CH₂—N(CH₃)—CH₂—CH₂— | * | 2-Chloro-6-fluorophenyl | —CH₃ | 0 | Cl | 1.47 | |
| 95 | —CH₂—CH₂—CH₂—CH₂— | * | 2-Chloro-6-fluorophenyl | —CH₃ | 0 | Cl | 3.52 | |
| 96 | —CH₂—CN | —H | 2-Chloro-6-fluorophenyl | —CH₃ | 0 | Cl | 2.47 | |
| 97 | —CH₂—CH₂—CH₂—CH₂—CH₂— | * | 2-Chloro-6-fluorophenyl | —CH₃ | 0 | Cl | 4.2 | |
| 98 | n-Propyl | —H | 2-Chloro-6-fluorophenyl | —CH₃ | 0 | Cl | 3.37 | |
| 99 | Cyclopentyl | —H | 2-Chloro-6-fluorophenyl | —CH₃ | 0 | Cl | 3.95 | |
| 100 | —C₂H₅ | —C₂H₅ | 2-Chloro-6-fluorophenyl | —CH₃ | 0 | Cl | 3.97 | |
| 101 | 2-Methoxyethyl | —H | 2-Chloro-6-fluorophenyl | —CH₃ | 0 | Cl | 2.89 | |
| 102 | Cyclopropyl | —H | 2-Chloro-6-fluorophenyl | —CH₃ | 0 | Cl | 3.16 | |
| 103 | —CH₂—CH₂—S—CH₂—CH₂— | * | 2-Chloro-6-fluorophenyl | —CH₃ | 0 | Cl | 3.75 | |
| 104 | —CH₂—CF₃ | —H | 2-Chloro-6-fluorophenyl | —CH₃ | 0 | Cl | 3.22 | |
| 105 | —CH₂—CH₂—CH(CF₃)—CH₂—CH₂— | * | 2-Chloro-6-fluorophenyl | —CH₃ | 0 | Cl | 4.22 | |
| 106 | Cyclopropylmethyl | —H | 2-Chloro-6-fluorophenyl | —CH₃ | 0 | Cl | 3.47 | |
| 107 | —CH₂—C(CH₃)═CH₂ | —H | 2-Chloro-6-fluorophenyl | —CH₃ | 0 | Cl | 3.38 | |
| 108 | —CH₂—CH₂—CF₃ | —H | 2-Chloro-6-fluorophenyl | —CH₃ | 0 | Cl | 3.38 | |
| 109 | 1-Cyclohexylethyl | —H | 2-Chloro-6-fluorophenyl | —CH₃ | 0 | Cl | 4.97 | |
| 110 | Cyclohexyl | —H | 2-Chloro-6-fluorophenyl | —CH₃ | 0 | Cl | 4.24 | |
| 111 | 2-Butyl | —H | 2-Chloro-6-fluorophenyl | —CH₃ | 0 | Cl | 3.74 | |
| 112 | N-Morpholinyl | —H | 2-Chloro-6-fluorophenyl | —CH₃ | 0 | Cl | 2.73 | |
| 113 | Pyrrolidin-1-yl | —H | 2-chloro-6-fluorophenyl | —CH₃ | 0 | Cl | 3.58 | |
| 114 | 1-Cyclopropylethylamino | —H | 2-Chloro-6-fluorophenyl | —CH₃ | 0 | Cl | 3.76 | |
| 115 | AB2 | —H | 2-chloro-6-fluorophenyl | —CH₃ | 0 | Cl | 4.45 | |
| 116 | i-Propylamino | —H | 2,4,6-Trifluorophenyl | —CH₃ | 0 | Cl | 3.29 | |
| 117 | 1-Butylamino | —H | 2,4,6-Trifluorophenyl | —CH₃ | 2 | Cl | 3.44 | |
| 118 | —CH₂—CH₂—CH═C(CH₃)—CH₂— | * | 2,4,6-Trifluorophenyl | —CH₃ | 0 | Cl | 4.19 | |
| 119 | —CH₂—CH₂—CH═CH—CH₂— | * | 2,4,6-Trifluorophenyl | —CH₃ | 0 | Cl | 3.78 | |
| 120 | —CH₃ | —CH₃ | Pentafluorophenyl | —CH₃ | 0 | Cl | 3.54 | |
| 121 | —CH₂—CH(CH₃)—O—CH(CH₃)—CH₂— | * | Pentafluorophenyl | —CH₃ | 0 | Cl | 4.15 | |
| 122 | —C₂H₅ | —H | Pentafluorophenyl | —CH₃ | 0 | Cl | 3.51 | |
| 123 | —CH₂—CH₂—O—CH₂—CH₂— | * | Pentafluorophenyl | —CH₃ | 0 | Cl | 3.46 | |
| 124 | 2-Methoxyethyl | —H | Pentafluorophenyl | —CH₃ | 0 | Cl | 3.4 | |
| 125 | —CH₃ | —H | Pentafluorophenyl | —CH₃ | 0 | Cl | 3.15 | |
| 126 | —CH₂—CH₂—S—CH₂—CH₂— | * | Pentafluorophenyl | —CH₃ | 0 | Cl | 4.08 | |
| 127 | n-Propylamino | —H | Pentafluorophenyl | —CH₃ | 0 | Cl | 3.86 | |
| 128 | AB2 | —H | Pentafluorophenyl | —CH₃ | 0 | Cl | 4.97 | |
| 129 | —NH—CH₂—CF₂—CHF₂ | —H | Pentafluorophenyl | —CH₃ | 0 | Cl | 3.56 | |
| 130 | —NH—CH₂—CF₃ | —H | Pentafluorophenyl | —CH₃ | 0 | Cl | 3.55 | |
| 131 | i-Butoxy | —H | 2,6-Difluorophenyl | —CH₃ | 0 | Cl | 3.5 | |
| 132 | —O—C₂H₅ | —H | 2,6-Difluorophenyl | —CH₃ | 0 | Cl | 2.77 | |
| 133 | 3-Chlorobenzyloxy | —H | 2,6-Difluomphenyl | —CH₃ | 0 | Cl | 3.8 | |
| 134 | 4-Chlorobenzyloxy | —H | 2,6-Difluorophenyl | —CH₃ | 0 | Cl | 3.81 | |
| 135 | 4-Fluorobenzyloxy | —H | 2,6-Difluorophenyl | —CH₃ | 0 | Cl | 3.47 | |
| 136 | —O—CH(CH₃)—CH₂—CH₃ | —H | 2,6-Difluorophenyl | —CH₃ | 0 | Cl | 3.39 | |
| 137 | Allyloxy | —H | 2,6-Difluorophenyl | —CH₃ | 0 | Cl | 2.93 | |
| 138 | t-Butoxy | —H | 2,6-Difluorophenyl | —CH₃ | 0 | Cl | 3.3 | |
| 139 | —O—CH₃ | —H | 2,6-Difluorophenyl | —CH₃ | 0 | Cl | 2.52 | |
| 140 | —O—CH₃ | —CH₃ | 2,6-Difluorophenyl | —CH₃ | 6 | Cl | 3.19 | |
| 141 | Benzyloxy | —H | 2,4,6-Trifluorophenyl | —CH₃ | 0 | Cl | 3.68 | |
| 142 | 3,5-Dichlorobenzyloxy | —H | 2,4,6-Trifluorophenyl | —CH₃ | 0 | Cl | 4.43 | |
| 143 | 2-Chlorobenzyloxy | —H | 2,4,6-Trifluorophenyl | —CH₃ | 0 | Cl | 3.94 | |
| 144 | 4-Chlorobenzyloxy | —H | 2,4,6-Trifluorophenyl | —CH₃ | 0 | Cl | 4 | |
| 145 | 4-Fluorobenzyloxy | —H | 2,4,6-Trifluorophenyl | —CH₃ | 6 | Cl | 3.65 | |
| 146 | -n-Butoxy | —H | 2,4,6-Trifluorophenyl | —CH₃ | 6 | Cl | 3.73 | |
| 147 | 2,6-Dichlorobenzyloxy | —H | 2,4,6-Trifluorophenyl | —CH₃ | 0 | Cl | 4.07 | |
| 148 | —O—CH₃ | —CH₃ | 2,4,6-Trifluorophenyl | —CH₃ | 0 | Cl | 3.43 | |
| 149 | —CH₂—CH(CH₃)—O—CH(CH₃)—CH₂— | * | 2,4,6-Trifluorophenyl | —CH₃ | 0 | Cl | 3.7 | |
| 150 | —CH₃ | —CH₃ | 2-Fluorophenyl | —CH₃ | 0 | Cl | | |
| 151 | —CH₂—CH₂—CH₂—CH₂— | * | 2-Fluorophenyl | —CH₃ | 0 | Cl | | |
| 152 | —CH₂—CH₂—CH₂—CH₂—CH₂— | * | 2-Fluorophenyl | —CH₃ | 6 | Cl | | |
| 153 | n-Propyl | —H | 2-Fluorophenyl | —CH₃ | 6 | Cl | | |
| 154 | Cyclopentyl | —H | 2-Fluorophenyl | —CH₃ | 6 | Cl | | |
| 155 | -i-Propyl | —H | 2-Fluorophenyl | —CH₃ | 0 | Cl | | |
| 156 | —C₂H₅ | —C₂H₅ | 2-Fluorophenyl | —CH₃ | 0 | Cl | | |
| 157 | 2-Methoxyethyl | —H | 2-Fluorophenyl | —CH₃ | 0 | Cl | | |
| 158 | —CH₃ | —H | 2-Fluorophenyl | —CH₃ | 0 | Cl | | |
| 159 | Cyclopropyl | —H | 2-Fluorophenyl | —CH₃ | 0 | Cl | | |
| 160 | —CH₂—CH₂—CH(CF₃)—CH₂—CH₂— | * | 2-Fluorophenyl | —CH₃ | 0 | Cl | | |
| 161 | Cyclopropylmethyl | —H | 2-Fluorophenyl | —CH₃ | 0 | Cl | | |
| 162 | 2,2,2-Trifluoro-1-methylethyl | —H | 2-Fluorophenyl | —CH₃ | 0 | Cl | | |
| 163 | —CH₂—C(CH₃)═CH₂ | —H | 2-Fluorophenyl | —CH₃ | 0 | Cl | | |
| 164 | —CH₂—CH₂—CF₃ | —H | 2-Fluorophenyl | —CH₃ | 0 | Cl | | |
| 165 | 1-Cyclohexylethyl | —H | 2-Fluorophenyl | —CH₃ | 0 | Cl | | |
| 166 | Cyclohexyl | —H | 2-Fluorophenyl | —CH₃ | 0 | Cl | | |
| 167 | 2-Butyl | —H | 2-Fluorophenyl | —CH₃ | 0 | Cl | | |
| 168 | —NH—CH₂—CH(CH₃)₂ | —H | 2,6-Dichloro-3-fluoro-5- | —CH₃ | 0 | Cl | 4.6 | |
| | | | trifluoromethylphenyl | | | | | |
| 169 | i-Butoxy | —H | 2-Fluorophenyl | —CH₃ | 0 | Cl | 3.52 | |
| 170 | —O—C₂H₅ | —H | 2-Fluorophenyl | —CH₃ | 0 | Cl | 2.76 | |
| 171 | 2-Chlorobenzyloxy | —H | 2-Fluorophenyl | —CH₃ | 0 | Cl | 3.78 | |
| 172 | 3-Chlorobenzyloxy | —H | 2-Fluorophenyl | —CH₃ | 0 | Cl | 3.83 | |
| 173 | 4-Chlorobenzyloxy | —H | 2-Fluorophenyl | —CH₃ | 0 | Cl | 3.85 | |
| 174 | 4-Fluorobenzyloxy | —H | 2-Fluorophenyl | —CH₃ | 0 | Cl | 3.48 | |
| 175 | —O—CH(CH₃)—CH₂—CH₃ | —H | 2-Fluorophenyl | —CH₃ | 0 | Cl | 3.42 | |
| 176 | Allyloxy | —H | 2-Fluorophenyl | —CH₃ | 0 | Cl | 2.92 | |
| 177 | t-Butoxy | —H | 2-Fluorophenyl | —CH₃ | 0 | Cl | 3.33 | |
| 178 | —O—CH₃ | —H | 2-Fluorophenyl | —CH₃ | 0 | Cl | 2.49 | |
| 179 | —O—CH₃ | —CH₃ | 2-Fluorophenyl | —CH₃ | 0 | Cl | 3.09 | |
| 180 | O-i-Propyl | —H | 2-Fluorophenyl | —CH₃ | 0 | Cl | 3.05 | |
| 181 | —CH₂—C(CH₃)═CH₂— | —C₂H₅ | 2,5-Difluorophenyl | —CH₃ | 0 | Cl | 4.25 | |
| 182 | —NH₂ | i-Butyl | 2,6-Dichloro-3-fluoro-5- | —CH₃ | 0 | Cl | 4.46 | |
| | | | trifluoromethylphenyl | | | | | |
| 183 | 3-Trifluoromethylbenzyloxy | —H | 2,4,6-Trifluorophenyl | —CH₃ | 0 | Cl | 4.04 | |
| 184 | Cyclohexyl | —H | 2,4,6-Trifluorophenyl | —CH₃ | 0 | Cl | 4.18 | |
| 185 | —CH₂—CH(CH₃)—O—CH(CH₃)—CH₂— | * | 2,6-Difluorophenyl | —CH₃ | 0 | Cl | 3.53 | |
| 186 | —CH₂—CH₂—CH₂—CH₂— | * | 2,6-Difluorophenyl | —CH₃ | 0 | Cl | 3.26 | |
| 187 | —CH₂—CN | —H | 2,6-Difluorophenyl | —CH₃ | 0 | Cl | 2.29 | |
| 188 | —CH₂—CH₂—CH₂—CH₂—CH₂— | * | 2,6-Difluorophenyl | —CH₃ | 0 | Cl | 3.9 | |
| 189 | n-Propyl | —H | 2,6-Difluorophenyl | —CH₃ | 0 | Cl | 3.15 | |
| 190 | Cyclopentyl | —H | 2,6-Difluorophenyl | —CH₃ | 0 | Cl | 3.69 | |
| 191 | -i-Propyl | —H | 2,6-Difluorophenyl | —CH₃ | 0 | Cl | 3.18 | |
| 192 | —C₂H₅ | —C₂H₅ | 2,6-Difluorophenyl | —CH₃ | 0 | Cl | 3.7 | |
| 193 | —CH₂—CH₂—O—CH₂—CH₂— | * | 2,6-Difluorophenyl | —CH₃ | 0 | Cl | 2.8 | |
| 194 | 2-Methoxyethyl | —H | 2,6-Difluorophenyl | —CH₃ | 0 | Cl | 2.68 | |
| 195 | Cyclopropyl | —H | 2,6-Difluorophenyl | —CH₃ | 0 | Cl | 2.91 | |
| 196 | —CH₂—CH₂—S—CH₂—CH₂— | * | 2,6-Difluorophenyl | —CH₃ | 0 | Cl | 3.49 | |
| 197 | —CH₂—CF₃ | —H | 2,6-Difluorophenyl | —CH₃ | 0 | Cl | 3.01 | |
| 198 | —CH₂—CH₂—CH(CF₃)—CH₂—CH₂— | * | 2,6-Difluorophenyl | —CH₃ | 0 | Cl | 3.97 | |
| 199 | Cyclopropylmethyl | —H | 2,6-Difluorophenyl | —CH₃ | 0 | Cl | 3.24 | |
| 200 | —CH₂—C(CH₃)═CH₂ | —H | 2,6-Difluorophenyl | —CH₃ | 0 | Cl | 3.15 | |
| 201 | —CH₂—CH₂—CF₃ | —H | 2,6-Difluorophenyl | —CH₃ | 0 | Cl | 3.18 | |
| 202 | 1-Cyclohexylethyl | —H | 2,6-Difluorophenyl | —CH₃ | 0 | Cl | 4.7 | |
| 203 | Cyclohexyl | —H | 2,6-Difluorophenyl | —CH₃ | 0 | Cl | 3.97 | |
| 204 | 2-Butyl | —H | 2,6-Difluorophenyl | —CH₃ | 0 | Cl | 3.49 | |
| 205 | 3-Trifluoromethylcyclohexyl | —H | 2,6-Difluorophenyl | —CH₃ | 0 | Cl | 3.98 | |
| 206 | 2-Trifluoromethylcyclohexyl | —H | 2,6-Difluorophenyl | —CH₃ | 0 | Cl | 4.06 | |
| 207 | 3,5-bis-Trifluoromethylcyclohexyl | —H | 2,6-Difluorophenyl | —CH₃ | 0 | Cl | 4.14 | |
| 208 | 4-Trifluoromethylcyclohexyl | —H | 2,6-Difluorophenyl | —CH₃ | 0 | Cl | 4 | |
| 209 | —CH₂—C(CH₃)═CH₂ | —C₂H₅ | 2,4-Difluorophenyl | —CH₃ | 0 | Cl | 4.36 | |
| 210 | —CH₃ | —CH₃ | 2,4,6-Trifluorophenyl | —CH₃ | 0 | Cl | 3.15 | |
| 211 | —C₂H₅ | —H | 2,4,6-Trifluorophenyl | —CH₃ | 0 | Cl | 3.03 | |
| 212 | —CH₃ | —H | 2,4,6-Trifluorophenyl | —CH₃ | 0 | Cl | 2.68 | |
| 213 | —CH₂—CH₂—CH═CH—CH₂— | * | 2-Chloro-6-fluorophenyl | —CH₃ | 0 | Cl | 3.89 | |
| 214 | 2-Hydroxypropyl | —H | 2-Chloro-6-fluorophenyl | —CH₃ | 0 | Cl | 2.38 | |
| 215 | —NH₂ | n-Propyl | 2,4,6-Trifluorophenyl | —CH₃ | 0 | Cl | 3.28 | 210-11 |
| 216 | —CH₂—C(CH₃)═CH₂ | —C₂H₅ | 2-Chloro-6-fluorophenyl | —CH₃ | 0 | Cl | 4.56 | |
| 217 | —NH—CH₂—CH(CH₃)₂ | —H | 2,6-Dichlorophenyl | —CH₃ | 0 | Cl | 3.96 | 213-15 |
| 219 | —NH₂ | i-Butyl | 4-Chloro-3-fluorophenyl | —CH₃ | 0 | Cl | 3.91 | 155-7 |
| 220 | —NH₂ | i-Butyl | 2,4-Difluoraphenyl | —CH₃ | 0 | Cl | 3.58 | 162-64 |
| 223 | —CH₂—C(CH₃)═CH₂— | —C₂H₅ | 2-Chlorophenyl | —CH₃ | 0 | Cl | 4.57 | |
| 224 | —CH₃ | —CH₃ | 2-Chloro-6-fluorophenyl | —CH₃ | 0 | Cl | 3.23 | |
| 225 | —CH₂—CH(CH₃)—O—CH(CH₃)—CH₂— | * | 2-Chloro-6-fluorophenyl | —CH₃ | 0 | Cl | 3.84 | |
| 226 | —C₂H₅ | —H | 2-Chloro-6-fluorophenyl | —CH₃ | 0 | Cl | 3.06 | |
| 227 | —C(CH₃)₂—CF₃ | —H | 2-Chloro-6-fluorophenyl | —CH₃ | 0 | Cl | 4.19 | |
| 228 | —CH₃ | —H | 2-Chloro-6-fluorophenyl | —CH₃ | 0 | Cl | 2.71 | |
| 229 | —CH(CF₃)—CH₂—CH₂—CH₂—CH₂— | * | 2-Chloro-6-fluorophenyl | —CH₃ | 0 | Cl | 4.43 | |
| 230 | 2,2,2-Trifluoro-1-methylethyl | —H | 2-Chloro-6-fluorophenyl | —CH₃ | 0 | Cl | 3.61 | |
| 231 | Dimethylamino | —H | 2-Chloro-6-fluorophenyl | —CH₃ | 0 | Cl | 3.18 | |
| 232 | —NH—CH₃ | —CH₃ | 2-Chloro-6-fluorophenyl | —CH₃ | 0 | Cl | 3.12 | |
| 233 | Ethylamino | —H | 2-Chloro-6-fluorophenyl | —CH₃ | 0 | Cl | 2.99 | |
| 234 | 1-Ethyl-1-propylamino | —H | 2-Chloro-6-fluorophenyl | —CH₃ | 0 | Cl | 4.08 | |
| 235 | n-Butylamino | —H | 2-Chloro-6-fluorophenyl | —CH₃ | 0 | Cl | 3.73 | |
| 236 | —NH—CH₂—CF₂—CHF₂ | —H | 2-Chloro-6-fluorophenyl | —CH₃ | 0 | Cl | 3.21 | |
| 237 | Allylamino | —H | 2-Chloro-6-fluorophenyl | —CH₃ | 0 | Cl | 3.19 | |
| 238 | —NH—CH₂—CF₃ | —H | 2-Chloro-6-fluorophenyl | —CH₃ | 0 | Cl | 3.12 | |
| 239 | —N(CH₃)—COOCH₃ | —H | 2-Chloro-6-fluorophenyl | —CH₃ | 0 | Cl | 2.47 | |
| 240 | 4-Trifluoromethylcyclohexyl | —H | 2,4,6-Trifluorophenyl | —CH₃ | 0 | Cl | 4.21 | |
| 241 | 2-Methoxyethyl | n-Propyl | 2,4,6-Trifluorophenyl | —CH₃ | 0 | Cl | 4.03 | |
| 242 | —CH₂—CH₂—NH₂ | -i-Propyl | 2,4,6-Trifluorophenyl | —CH₃ | 0 | Cl | 1.61 | |
| 243 | —CH₂—CH(OH)—CH₂—CH₂— | * | 2,4,6-Trifluorophenyl | —CH₃ | 0 | Cl | 2.38 | |
| 244 | —CH₂—CH₂—CH₂—CH(CH₃)— | * | 2,4,6-Trifluorophenyl | —CH₃ | 0 | Cl | 3.88 | |
| 245 | —CH₂—CH(NH₂)—CH₂—CH₂— | * | 2,4,6-Trifluorophenyl | —CH₃ | 0 | Cl | 1.51 | |
| 246 | AB8 | * | 2,4,6-Trifluorophenyl | —CH₃ | 0 | Cl | 2.22 | |
| 247 | AB9 | * | 2,4,6-Trifluorophenyl | —CH₃ | 0 | Cl | 1.56 | |
| 248 | —CH₂—CH₂—CH(OH)—CH₂—CH₂— | * | 2,4,6-Trifluorophenyl | —CH₃ | 0 | Cl | 2.5 | |
| 249 | AB10 | * | 2,4,6-Trifluorophenyl | —CH₃ | 0 | Cl | 3.43 | |
| 250 | —CH₂—CH₂—CH₂—CH₂—CH(CH₃)— | * | 2,4,6-Trifluorophenyl | —CH₃ | 0 | Cl | 4.45 | |
| 251 | —CH₂—CH₂—CH₂—CH(CH₃)—CH₂— | * | 2,4,6-Trifluorophenyl | —CH₃ | 0 | Cl | 4.51 | |
| 252 | —CH₂—CH₂—CH(CH₃)—CH₂—CH₂— | * | 2,4,6-Trifluorophenyl | —CH₃ | 0 | Cl | 4.51 | |
| 253 | —CH₂—CH₂—C(CH₃)₂—CH₂—CH₂— | * | 2,4,6-Trifluorophenyl | —CH₃ | 0 | Cl | 4.78 | |
| 254 | AB11 | * | 2,4,6-Trifluorophenyl | —CH₃ | 0 | Cl | 1.68 | |
| 255 | —CH₂—CH₂—N(CH₃)₂ | —CH₃ | 2,4,6-Trifluorophenyl | —CH₃ | 0 | Cl | 1.64 | |
| 256 | 3-(Dimethylamino)-propyl | —CH₃ | 2,4,6-Trifluorophenyl | —CH₃ | 0 | Cl | 1.74 | |
| 257 | —CH₂—CH₂—N(CH₃)₂ | —C₂H₅ | 2,4,6-Trifluorophenyl | —CH₃ | 0 | Cl | 1.82 | |
| 258 | —CH₂CH₂OH | —C₂H₅ | 2,4,6-Tiifluorophenyl | —CH₃ | 0 | Cl | 2.32 | |
| 259 | —CH₂—CH₂—NH₂ | —C₂H₅ | 2,4,6-Trifluorophenyl | —CH₃ | 0 | Cl | 1.54 | |
| 260 | —CH₂—CH₂—NH₂ | n-Propyl | 2,4,6-Trifluorophenyl | —CH₃ | 0 | Cl | 1.66 | |
| 261 | 3-Aminopropyl | n-Propyl | 2,4,6-Trifluorophenyl | —CH₃ | 0 | Cl | 1.72 | |
| 262 | Benzyloxy | —H | 2,6-Difluorophenyl | —CH₃ | 0 | Cl | 3.49 | |
| 263 | 3,5-Dichlorobenzyloxy | —H | 2,6-Difluorophenyl | —CH₃ | 0 | Cl | 4.33 | |
| 264 | 4-Trifluoromethylbenzyloxy | —H | 2,6-Difluorophenyl | —CH₃ | 0 | Cl | 3.97 | |
| 265 | 2-Chlorobenzyloxy | —H | 2,6-Difluorophenyl | —CH₃ | 0 | Cl | 3.77 | |
| 266 | 3-Trifluoromethylbenzyloxy | —H | 2,6-Difluorophenyl | —CH₃ | 0 | Cl | 3.94 | |
| 267 | -n-Butoxy | —H | 2,6-Difluorophenyl | —CH₃ | 0 | Cl | 3.53 | |
| 268 | 2,6-Dichlorobenzyloxy | —H | 2,6-Difluorophenyl | —CH₃ | 0 | Cl | 3.92 | |
| 269 | Benzyloxy | —H | 2-Fluorophenyl | —CH₃ | 0 | Cl | 3.49 | |
| 270 | 3,5-Dichlorobenzyloxy | —H | 2-Fluorophenyl | —CH₃ | 0 | Cl | 4.37 | |
| 271 | —O-n-Propyl | —H | 2-Fluorophenyl | —CH₃ | 0 | Cl | 4.35 | |
| 272 | 4-Trifluoromethylbenzyloxy | —H | 2-Fluorophenyl | —CH₃ | 0 | Cl | 4.02 | |
| 273 | 3-Trifluoromethylbenzyloxy | —H | 2-Fluorophenyl | —CH₃ | 0 | Cl | 3.99 | |
| 274 | -n-Butoxy | —H | 2-Fluorophenyl | —CH₃ | 0 | Cl | 3.57 | |
| 275 | 2-Hexahydropyranyloxy | —H | 2-Fluorophenyl | —CH₃ | 0 | Cl | 3.2 | |
| 276 | 2,6-Dichlorobenzyloxy | —H | 2-Fluorophenyl | —CH₃ | 0 | Cl | 4.03 | |
| 277 | i-Butyl | —H | 2,4,6-Trifluorophenyl | —CH₃ | 0 | Cl | 3.66 | |
| 278 | n-Butyl | —H | 2,4,6-Trifluorophenyl | —CH₃ | 0 | Cl | 3.71 | |
| 279 | —CH₂—C(CH₃)₃ | —H | 2,4,6-Trifluorophenyl | —CH₃ | 0 | Cl | 4.06 | |
| 280 | Propargyl | —CH₃ | 2,4,6-Trifluorophenyl | —CH₃ | 0 | Cl | 3.28 | |
| 281 | 1,3-Dioxolan-2-ylmethyl | —CH₃ | 2,4,6-Trifluorophenyl | —CH₃ | 0 | Cl | 3.23 | |
| 282 | Allyl | —CH₃ | 2,4,6-Trifluorophenyl | —CH₃ | 0 | Cl | 3.76 | |
| 283 | —CH₂—CH₂—CN | —CH₃ | 2,4,6-Trifluorophenyl | —CH₃ | 0 | Cl | 2.83 | |
| 284 | —CH₂—CH(OCH₃)₂ | —CH₃ | 2,4,6-Trifluorophenyl | —CH₃ | 0 | Cl | 3.36 | |
| 285 | (2-Furyl)methyl | —CH₃ | 2,4,6-Trifluorophenyl | —CH₃ | 0 | Cl | 3.77 | |
| 286 | i-Butyl | —CH₃ | 2,4,6-Trifluorophenyl | —CH₃ | 0 | Cl | 4.25 | |
| 287 | 2-Methoxyethyl | —CH₃ | 2,4,6-Trifluorophenyl | —CH₃ | 0 | Cl | 3.27 | |
| 288 | —CH₂—C(CH₃)═CH₂ | —CH₃ | 2,4,6-Trifluorophenyl | —CH₃ | 0 | Cl | 4.11 | |
| 289 | n-Butyl | —CH₃ | 2,4,6-Trifluorophenyl | —CH₃ | 0 | Cl | 4.31 | |
| 290 | i-Butyl | —H | Pentafluorophenyl | —CH₃ | 0 | Cl | 4.09 | |
| 291 | —CH₂—CH₂—CF₃ | —H | Pentafluorophenyl | —CH₃ | 0 | Cl | 3.74 | |
| 292 | Cyclohexyl | —H | Pentafluorophenyl | —CH₃ | 0 | Cl | 4.59 | |
| 293 | 1-cyclohexylethyl | —H | Pentafluorophenyl | —CH₃ | 0 | Cl | 5.23 | |
| 294 | —CH₂—C(CH₃)═CH₂ | —H | Pentafluorophenyl | —CH₃ | 0 | Cl | 3.78 | |
| 295 | 4-Trifluoromethylcyclohexyl | —H | Pentafluorophenyl | —CH₃ | 0 | Cl | 4.53 | |
| 296 | —CH(CH₃)—CH₂—CH(CH₃)₂ | —H | Pentafluorophenyl | —CH₃ | 0 | Cl | 4.76 | |
| 297 | —CH₂—CH₂—N(CH₃)₂ | —CH₃ | Pentafluorophenyl | —CH₃ | 0 | Cl | 1.82 | |
| 298 | 1,3-Dioxolan-2-ylmethyl | —CH₃ | Pentafluorophenyl | —CH₃ | 0 | Cl | 3.64 | |
| 299 | —CH₂—CH₂—CN | —CH₃ | Pentafluorophenyl | —CH₃ | 0 | Cl | 3.19 | |
| 300 | —CH₂—CH(OCH₃)₂ | —CH₃ | Pentafluorophenyl | —CH₃ | 0 | Cl | 3.78 | |
| 301 | (2-Furyl)methyl | —CH₃ | Pentafluorophenyl | —CH₃ | 0 | Cl | 4.11 | |
| 302 | i-Butyl | —CH₃ | Pentafluorophenyl | —CH₃ | 0 | Cl | 4.58 | |
| 303 | 2-Methoxyethyl | —CH₃ | Pentafluorophenyl | —CH₃ | 0 | Cl | 3.69 | |
| 304 | —CH₂—C(CH₃)═CH₂— | —CH₃ | Pentafluorophenyl | —CH₃ | 0 | Cl | 4.43 | |
| 305 | —CH₂—CH₂—N(CH₃)₂ | —C₂H₅ | Pentafluorophenyl | —CH₃ | 0 | Cl | 1.97 | |
| 306 | (2-Tetrahydrofuxyl)methyl | —C₂H₅ | Pentafluorophenyl | —CH₃ | 0 | Cl | 4.31 | |
| 307 | 2-Methoxyethyl | —C₂H₅ | Pentafluorophenyl | —CH₃ | 0 | Cl | 4.05 | |
| 308 | —CH₂—CH(CH₃)—O—CH(CH₃)—CH₂— | * | 2-Fluorophenyl | —CH₃ | 0 | Cl | 3.5 | |
| 309 | —C₂H₅ | —H | 2-Fluorophenyl | —CH₃ | 0 | Cl | 2.83 | |
| 310 | —CH₂—CN | —H | 2-Fluorophenyl | —CH₃ | 0 | Cl | 2.27 | |
| 311 | —CH₂—CH₂—O—CH₂—CH₂— | * | 2-Fluorophenyl | —CH₃ | 0 | Cl | 2.78 | |
| 312 | —C(CH₃)₂—CH₂—COCH₃ | —H | 2-Fluorophenyl | —CH₃ | 0 | Cl | 3.2 | |
| 313 | —C₂H₅ | —H | 2,6-Difluorophenyl | —CH₃ | 0 | Cl | 2.86 | |
| 314 | —C(CH₃)₂—CF₃ | —H | 2,6-Difluorophenyl | —CH₃ | 0 | Cl | 4.05 | |
| 315 | —CH₃ | —H | 2,6-Difluorophenyl | —CH₃ | 0 | Cl | 2.53 | |
| 316 | —CH(CF₃)—CH₂—CH₂—CH₂—CH₂— | * | 2,6-Difluorophenyl | —CH₃ | 0 | Cl | 4.05 | |
| 317 | 2,2,2-Trifluoro-1-methylethyl | —H | 2,6-Difluorophenyl | —CH₃ | 0 | Cl | 3.27 | |
| 318 | —CH₃ | —CH₃ | 2,6-Difluorophenyl | —CH₃ | 0 | Cl | 3 | |
| 319 | i-Butoxy | —H | Pentafluorophenyl | —CH₃ | 0 | Cl | 4.17 | |
| 320 | —O—C₂H₅ | —H | Pentafluorophenyl | —CH₃ | 0 | Cl | 3.43 | |
| 321 | Benzyloxy | —H | Pentafluorophenyl | —CH₃ | 0 | Cl | 4.11 | |
| 322 | 3,5-Dichlorobenzyloxy | —H | Pentafluorophenyl | —CH₃ | 0 | Cl | 4.75 | |
| 323 | 2,4-Dichlorobenzyloxy | —H | Pentafluorophenyl | —CH₃ | 0 | Cl | 4.73 | |
| 324 | 4-Trifluoromethylbenzyloxy | —H | Pentafluorophenyl | —CH₃ | 0 | Cl | 4.41 | |
| 325 | 2-Chlorobenzyloxy | —H | Pentafluorophenyl | —CH₃ | 0 | Cl | 4.3 | |
| 326 | 3-Chlorobenzyloxy | —H | Pentafluorophenyl | —CH₃ | 0 | Cl | 4.33 | |
| 327 | 4-Chlorobenzyloxy | —H | Pentafluorophenyl | —CH₃ | 0 | Cl | 4.31 | |
| 328 | 4-Fluorobenzyloxy | —H | Pentafluorophenyl | —CH₃ | 0 | Cl | 4.04 | |
| 329 | —O—CH(CH₃)—CH₂—CH₃ | —H | Pentafluorophenyl | —CH₃ | 0 | Cl | 4.06 | |
| 330 | 3-Trifluoromethylbenzyloxy | —H | Pentafluorophenyl | —CH₃ | 0 | Cl | 4.37 | |
| 331 | -n-Butoxy | —H | Pentafluorophenyl | —CH₃ | 0 | Cl | 4.39 | |
| 332 | Allyloxy | —H | Pentafluorophenyl | —CH₃ | 0 | Cl | 3.59 | |
| 333 | t-Butoxy | —H | Pentafluorophenyl | —CH₃ | 0 | Cl | 3.98 | |
| 334 | 2-Hexahydropyranyloxy | —H | Pentafluorophenyl | —CH₃ | 0 | Cl | 3.82 | |
| 335 | 2,6-Dichlorobenzyloxy | —H | Pentafluorophenyl | —CH₃ | 0 | Cl | 4.23 | |
| 336 | —O—CH₃ | —H | Pentafluorophenyl | —CH₃ | 0 | Cl | 3.15 | |
| 337 | —O—CH₃ | —CH₃ | Pentafluorophenyl | —CH₃ | 0 | Cl | 3.85 | |
| 338 | O-i-Propyl | —H | Pentafluorophenyl | —CH₃ | 0 | Cl | 3.71 | |
| 339 | 2,2,2-Trifluoro-1-methylethyl | —H | 2,4-Difluorophenyl | —CH₃ | 0 | Cl | 3.52 | |
| 340 | 1-Cyclopropylethyl | —H | 2,4,6-Trifluorophenyl | —CH₃ | 0 | Cl | 3.71 | |
| 341 | i-Butyl | —H | 2,6-Difluorophenyl | —CH₃ | 0 | Cl | 3.46 | |
| 342 | n-Butyl | —H | 2,6-Difluorophenyl | —CH₃ | 0 | Cl | 3.5 | |
| 343 | —CH₂—C(CH₃)₃ | —H | 2,6-Difluorophenyl | —CH₃ | 0 | Cl | 3.88 | |
| 344 | —CH(CH₃)—CH₂—CH(CH₃)₂ | —H | 2,6-Difluorophenyl | —CH₃ | 0 | Cl | 4.19 | |
| 345 | Propargyl | —CH₃ | 2,6-Difluorophenyl | —CH₃ | 0 | Cl | 3.1 | |
| 346 | Allyl | —CH₃ | 2,6-Difluorophenyl | —CH₃ | 0 | Cl | 3.59 | |
| 347 | —CH₂—CH₂—CN | —CH₃ | 2,6-Difluorophenyl | —CH₃ | 0 | Cl | 2.66 | |
| 348 | —CH₂—CH(OCH₃)₂ | —CH₃ | 2,6-Difluorophenyl | —CH₃ | 0 | Cl | 3.16 | |
| 349 | (2-Furyl)methyl | —CH₃ | 2,6-Difluorophenyl | —CH₃ | 0 | Cl | 3.58 | |
| 350 | i-Butyl | —CH₃ | 2,6-Difluorophenyl | —CH₃ | 0 | Cl | 4.06 | |
| 351 | 2-Methoxyethyl | —CH₃ | 2,6-Difluorophenyl | —CH₃ | 0 | Cl | 3.08 | |
| 352 | —CH₂—C(CH₃)═CH₂ | —CH₃ | 2,6-Difluorophenyl | —CH₃ | 0 | Cl | 3.94 | |
| 353 | n-Butyl | —CH₃ | 2,6-Difluorophenyl | —CH₃ | 0 | Cl | 4.13 | |
| 354 | —CH₂—CH₂—N(CH₃)₂ | —C₂₅ | 2,6-Difluorophenyl | —CH₃ | 0 | Cl | 1.66 | |
| 355 | Allyl | —C₂H₅ | 2,6-Difluorophenyl | —CH₃ | 0 | Cl | 3.91 | |
| 356 | (2-Furyl)methyl | —C₂H₅ | 2,6-Difluorophenyl | —CH₃ | 0 | Cl | 3.89 | |
| 357 | (2-Tetrahydrofinyl)methyl | —C₂H₅ | 2,6-Difluorophenyl | —CH₃ | 0 | Cl | 3.67 | |
| 358 | —CH₂—CH₂—CN | —C₂H₅ | 2,6-Difluorophenyl | —CH₃ | 0 | Cl | 2.93 | |
| 359 | 2-Methoxyethyl | —C₂H₅ | 2,6-Difluorophenyl | —CH₃ | 0 | Cl | 3.4 | |
| 360 | —CH₂—COOC₂H₅ | —C₂H₅ | 2,6-Difluorophenyl | —CH₃ | 0 | Cl | 3.38 | |
| 361 | n-Butyl | —C₂H₅ | 2,6-Difluorophenyl | —CH₃ | 0 | Cl | 4.47 | |
| 362 | —CH₂—CH₂OH | n-Propyl | 2,6-Difluorophenyl | —CH₃ | 0 | Cl | 2.65 | |
| 363 | 3-Aminopropyl | n-Propyl | 2,6-Difluorophenyl | —CH₃ | 0 | Cl | 1.55 | |
| 364 | Cyclopropylmethyl | n-Propyl | 2,6-Difluorophenyl | —CH₃ | 0 | Cl | 4.49 | |
| 365 | (2-Tetrahydropyranyl)methyl | n-Propyl | 2,6-Difluorophenyl | —CH₃ | 0 | Cl | 4.64 | |
| 366 | (2-Tetrahydrofunyl)methyl | n-Propyl | 2,6-Difluorophenyl | —CH₃ | 0 | Cl | 4.06 | |
| 367 | 2,2-Diethoxy-ethyl | n-Butyl | 2,4-Difluorophenyl | —CH₃ | 0 | Cl | 5.03 | |
| 368 | 2,2,2-Trifluoro-1-methylethyl | —H | 2,5-Difluorophenyl | —CH₃ | 0 | Cl | 3.43 | |
| 369 | —O—CH₃ | —H | 2,4-Difluorophenyl | —CH₃ | 0 | Cl | 2.66 | |
| 370 | n-Butyl | —C₂H₅ | Pentafluorophenyl | —CH₃ | 0 | Cl | 4.98 | |
| 371 | cyclopropylmethyl | n-Propyl | Pentafluorophenyl | —CH₃ | 0 | Cl | 5.01 | |
| 372 | (2-Tetrahydropyranyl)methyl | n-Propyl | Pentafluorophenyl | —CH₃ | 0 | Cl | 5.29 | |
| 373 | (2-Tetrahydrofuryl)methyl | n-Propyl | Pentafluorophenyl | —CH₃ | 0 | Cl | 4.72 | |
| 374 | 2-Methoxyethyl | n-Propyl | Pentafluorophenyl | —CH₃ | 0 | Cl | 4.42 | |
| 375 | —CH₂—CH(OH)—CH₂—CH₂— | * | Pentafluorophenyl | —CH₃ | 0 | Cl | 2.72 | |
| 376 | —CH₂—CH₂—CH₂—CH(CH₃)— | * | Pentafluorophenyl | —CH₃ | 0 | Cl | 4.21 | |
| 377 | AB8 | * | Pentafluorophenyl | —CH₃ | 0 | Cl | 2.56 | |
| 378 | AB9 | * | Pentafluorophenyl | —CH₃ | 0 | Cl | 1.79 | |
| 379 | —CH₂—CH₂—CH(OH)—CH₂—CH₂— | * | Pentafluorophenyl | —CH₃ | 0 | Cl | 2.84 | |
| 380 | n-Butyl | —H | 2,4-Difluorophenyl | —CH₃ | 0 | Cl | 3.62 | |
| 381 | —CH₂—COOC₂H₅ | —CH₃ | 2,4,6-Trifluorophenyl | —CH₃ | 0 | Cl | 3.31 | |
| 382 | —CH₂—CN | —CH₃ | 2,4,6-Trifluorophenyl | —CH₃ | 0 | Cl | 2.85 | |
| 383 | —CH₂—COOCH₃ | —CH₃ | 2,4,6-Trifluorophenyl | —CH₃ | 0 | Cl | 2.98 | |
| 384 | —CH₂—CH₂—Cl | —CH₃ | 2,4,6-Trifluorophenyl | —CH₃ | 0 | Cl | 3.61 | |
| 385 | —CH₂—CH₂—O—CH═CH₂ | —CH₃ | 2,4,6-Trifluorophenyl | —CH₃ | 0 | Cl | 2.45 | |
| 386 | 2-Butyl | —H | 2,4,6-Trifluorophenyl | —CH₃ | 0 | Cl | 3.74 | |
| 387 | —CH═C(CH₃)₂ | —C₂H₅ | 2,4,6-Trifluorophenyl | —CH₃ | 0 | Cl | 2.18 | |
| 388 | Allyl | —C₂H₅ | 2,4,6-Trifluorophenyl | —CH₃ | 0 | Cl | 4.14 | |
| 389 | (2-Furyl)methyl | —C₂H₅ | 2,4,6-Trifluorophenyl | —CH₃ | 0 | Cl | 4.14 | |
| 390 | (2-Tetrahydrofuryl)methyl | —C₂H₅ | 2,4,6-Trifluorophenyl | —CH₃ | 0 | Cl | 3.93 | |
| 391 | —CH₂—CH₂—CN | —C₂H₅ | 2,4,6-Trifluorophenyl | —CH₃ | 0 | Cl | 3.15 | |
| 392 | —CH₂—COOC₂H₅ | —C₂H₅ | 2,4,6-Trifluorophenyl | —CH₃ | 0 | Cl | 3.65 | |
| 393 | n-Butyl | —C₂H₅ | 2,4,6-Trifluorophenyl | —CH₃ | 0 | Cl | 4.7 | |
| 394 | —CH₂CH₂OH | n-Propyl | 2,4,6-Trifluorophenyl | —CH₃ | 0 | Cl | 2.96 | |
| 395 | Cyclopropylmethyl | n-Propyl | 2,4,6-Trifluorophenyl | —CH₃ | 0 | Cl | 4.73 | |
| 396 | (2-Tetrahydropyranyl)methyl | n-Propyl | 2,4,6-Trifluorophenyl | —CH₃ | 0 | Cl | 4.91 | |
| 397 | —CH₂—CH₂—COOC₂H₅ | n-Propyl | 2,4,6-Trifluorophenyl | —CH₃ | 0 | Cl | 3.47 | |
| 398 | (2-Tetrahydrofuryl)methyl | n-Propyl | 2,4,6-Trifluorophenyl | —CH₃ | 0 | Cl | 4.34 | |
| 399 | 2-Thienylmethyl | n-Propyl | 2,4,6-Trifluorophenyl | —CH₃ | 0 | Cl | 4.76 | |
| 400 | —C(CH₃)₂—CF₃ | —H | 2,4,6-Trifluorophenyl | —CH₃ | 0 | Cl | 4.07 | |
| 401 | 1-Cyclohexylethyl | —H | 2,4,6-Trifluorophenyl | —CH₃ | 0 | Cl | 4.93 | |
| 402 | 3-Trifluoromethylcyclohexyl | —H | 2,4,6-Trifluorophenyl | —CH₃ | 0 | Cl | 4.21 | |
| 403 | 2-Trifluoromethylcyclohexyl | —H | 2,4,6-Trifluorophenyl | —CH₃ | 0 | Cl | 4.31 | |
| 404 | —CH(CH₃)—CH₂—CH(CH₃)₂ | —H | 2,4,6-Trifluorophenyl | —CH₃ | 0 | Cl | 4.43 | |
| 405 | 3,5-bis-Trifluoromethylcyclohexyl | —H | 2,4,6-Trifluorophenyl | —CH₃ | 0 | Cl | 4.37 | |
| 406 | —CH₂—COOC₂H₅ | Cyclo- | 2,4,6-Trifluorophenyl | —CH₃ | 0 | Cl | 3.76 | |
| | | propyl | | | | | | |
| 407 | —CH(CF₃)—CH₂—CH₂—CH₂— | * | 2,4,6-Trifluorophenyl | —CH₃ | 0 | Cl | 4.07 | |
| 408 | —CH₂—CH₂—CO—CH₂—CH₂— | * | 2,4,6-Trifluorophenyl | —CH₃ | 0 | Cl | 2.84 | |
| 409 | —CH₂—CH(CH₃)—CH₂—CH(CH₃)—CH₂— | * | 2,4,6-Trifluorophenyl | —CH₃ | 0 | Cl | 4.87 | |
| 410 | —CH₂—CH(OH)—CH₂—CH₂—CH₂— | * | 2,4,6-Trifluorophenyl | —CH₃ | 0 | Cl | 2.74 | |
| 411 | —CH₂—CH₂—CH(COCH₃)—CH₂—CH₂— | * | 2,4,6-Trifluorophenyl | —CH₃ | 0 | Cl | 3.2 | |
| 412 | —CH₂—CH═C(C₂H₅)—CH₂—CH₂— | * | 2,4,6-Trifluorophenyl | —CH₃ | 0 | Cl | 3.85 | |
| 413 | —CH(CH₂—NH₂)—CH₂—CH₂—CH₂—CH₂— | * | 2,4,6-Trifluorophenyl | —CH₃ | 0 | Cl | 4.68 | |
| 414 | —CH₂—CH₂—CH(COOCH₃)—CH₂—CH₂— | * | 2,4,6-Trifluorophenyl | —CH₃ | 0 | Cl | 3.94 | |
| 415 | —CH₂—CH₂—CHBr—CH₂—CH₂— | * | 2,4,6-Trifluorophenyl | —CH₃ | 0 | Cl | 3.51 | |
| 416 | —CH(COOCH₃)—CH₂—CH₂—CH₂—CH₂— | * | 2,4,6-Trifluorophenyl | —CH₃ | 0 | Cl | 4.16 | |
| 417 | —CH₂—CH₂—CHF—CH₂—CH₂— | * | 2,4,6-Trifluorophenyl | —CH₃ | 0 | Cl | 3.61 | |
| 418 | AB12 | * | 2,4,6-Trifluorophenyl | —CH₃ | 0 | Cl | 3.98 | |
| 419 | —CH₂—CH₂—CH(NH—COCH₃)CH₂— | * | 2,4,6-Tzifluorophenyl | —CH₃ | 0 | Cl | 2.44 | |
| | CH₂— | | | | | | | |
| 420 | —CH(CF₃)—CH₂—CH₂—CH₂—CH₂— | * | 2,4,6-Trifluorophenyl | —CH₃ | 0 | Cl | 4.42 | |
| 421 | AB14 | * | 2,4,6-Trifluorophenyl | —CH₃ | 0 | Cl | 4.36 | |
| 422 | 3-Aminopropyl | n-Propyl | Pentafluorophenyl | —CH₃ | 0 | Cl | 1.93 | |
| 423 | 2-Thienylmethyl | n-Propyl | Pentafluorophenyl | —CH₃ | 0 | Cl | 5.05 | |
| 424 | —CH₂—CH₂—NH₂ | -i-Propyl | Pentafluorophenyl | —CH₃ | 0 | Cl | 1.83 | |
| 425 | —CH₂—CH₂—CN | -i-Propyl | Pentafluorophenyl | —CH₃ | 0 | Cl | 3.82 | |
| 426 | —CH₂—COOC₂H₅ | Cyclo- | Pentafluorophenyl | —CH₃ | 0 | Cl | 4.13 | |
| | | propyl | | | | | | |
| 427 | —CH(CF₃)—CH₂—CH₂—CH₂— | * | Pentafluorophenyl | —CH₃ | 0 | Cl | 4.39 | |
| 428 | —CH₂—CH₂—CH₂—CH₂— | * | Pentafluorophenyl | —CH₃ | 0 | Cl | 3.86 | |
| 429 | n-Butyl | —H | Pentafluorophenyl | —CH₃ | 0 | Cl | 4.19 | |
| 430 | —CH₂—C(CH₃)₃ | —H | Pentafluorophenyl | —CH₃ | 0 | Cl | 4.5 | |
| 431 | 2-Butyl | —H | Pentafluorophenyl | —CH₃ | 0 | Cl | 4.16 | |
| 432 | 3,5-bis-Trifluoromethylcyclohexyl | —H | Pentafluorophenyl | —CH₃ | 0 | Cl | 4.69 | |
| 433 | Propargyl | —CH₃ | Pentafluorophenyl | —CH₃ | 0 | Cl | 3.71 | |
| 434 | —CH₂—COOC₂H₅ | —CH₃ | Pentafluorophenyl | —CH₃ | 0 | Cl | 3.73 | |
| 435 | Allyl | —CH₃ | Pentafluorophenyl | —CH₃ | 0 | Cl | 4.15 | |
| 436 | —CH₂—CN | —CH₃ | Pentafluorophenyl | —CH₃ | 0 | Cl | 3.23 | |
| 437 | —CH₂—CH₂—Cl | —CH₃ | Pentafluorophenyl | —CH₃ | 0 | Cl | 3.92 | |
| 438 | —CH═C(CH₃)₂ | —C₂H₅ | Pentafluorophenyl | —CH₃ | 0 | Cl | 2.5 | |
| 439 | Allyl | —C₂H₅ | Pentafluorophenyl | —CH₃ | 0 | Cl | 4.46 | |
| 440 | (2-Furyl)methyl | —C₂H₅ | Pentafluorophenyl | —CH₃ | 0 | Cl | 4.48 | |
| 441 | —CH₂—CH₂—CN | —C₂H₅ | Pentafluorophenyl | —CH₃ | 0 | Cl | 3.5 | |
| 442 | —CH₂—COOC₂H₅ | —C₂H₅ | Pentafluorophenyl | —CH₃ | 0 | Cl | 4.07 | |
| 443 | —NH—CH₂—CH₂—CH₂—CH₂— | * | 2,4,6-Trifluorophenyl | —CH₃ | 0 | Cl | 3.45 | 175-7 |
| 444 | —CH₂—CH₂—CH(CH₃)—CH₂—CH₂— | * | 2-Chlorophenyl | —CH₃ | 0 | Cl | 4.69 | |
| 445 | 2,2,2-Trifluoro-1-methylethyl | —H | 2,6-Difluorophenyl | —CH₃ | 0 | Cl | 3.44 | |
| 446 | —CH₂—COOCH₃ | —CH₃ | 2,6-Difluorophenyl | —CH₃ | 0 | Cl | 2.8 | |
| 447 | 1,3-Dioxolan-2-ylmethyl | —CH₃ | 2,6-Difluorophenyl | —CH₃ | 0 | Cl | 3.1 | |
| 448 | —CH₂—CH₂—N(CH₃)₂ | —CH₃ | 2,6-Difluorophenyl | —CH₃ | 0 | Cl | 1.43 | |
| 449 | 3-Dimethylamino)-propyl | —CH₃ | 2,6-Difluorophenyl | —CH₃ | 0 | Cl | 2.81 | |
| 450 | —CH₂—COOC₂H₅ | —CH₃ | 2,6-Difluorophenyl | —CH₃ | 0 | Cl | 3.14 | |
| 451 | —CH₂—CH₂—Cl | —CH₃ | 2,6-Difluorophenyl | —CH₃ | 0 | Cl | 3.44 | |
| 452 | —CH₂—C(CH₃)═CH₂ | —C₂H₅ | 2,6-Difluorophenyl | —CH₃ | 0 | Cl | 4.27 | |
| 453 | 2-Methoxyethyl | n-Propyl | 2,6-Difluorophenyl | —CH₃ | 0 | Cl | 3.78 | |
| 454 | —CH₂—CH₂—NH₂ | -i-Propyl | 2,6-Difluorophenyl | —CH₃ | 0 | Cl | 1.47 | |
| 455 | —CH₂—CH(OH)—CH₂—CH₂— | * | 2,6-Difluorophenyl | —CH₃ | 0 | Cl | 2.17 | |
| 456 | —CH₂—CH₂—CH₂—CH(CH₃)— | * | 2,6-Difluorophenyl | —CH₃ | 0 | Cl | 3.63 | |
| 457 | AB8 | * | 2,6-Difluorophenyl | —CH₃ | 0 | Cl | 2.03 | |
| 458 | —CH(CF₃)—CH₂—CH₂—CH₂— | * | 2,6-Difluorophenyl | —CH₃ | 0 | Cl | 3.78 | |
| 459 | AB9 | * | 2,6-Difluorophenyl | —CH₃ | 0 | Cl | 1.42 | |
| 460 | —CH₂—CH₂—CH═CH—CH₂— | * | 2,6-Difluorophenyl | —CH₃ | 0 | Cl | 3.6 | |
| 461 | AB10 | * | 2,6-Difluorophenyl | —CH₃ | 0 | Cl | 3.19 | |
| 462 | —CH₂—CH(CH₃)—CH₂—CH(CH₃)—CH₂— | * | 2,6-Difluorophenyl | —CH₃ | 0 | Cl | 4.69 | |
| 463 | —CH₂—CH₂—CH₂—CH₂—CH(CH₃)— | * | 2,6-Difluorophenyl | —CH₃ | 0 | Cl | 4.2 | |
| 464 | —CH₂—CH₂—CH₂—CH(CH₃)—CH₂— | * | 2,6-Difluorophenyl | —CH₃ | 0 | Cl | 4.2 | |
| 465 | —CH₂—CH₂—CH(CH₃)—CH₂—CH₂— | * | 2,6-Difluorophenyl | —CH₃ | 0 | Cl | 4.27 | |
| 466 | —CH₂—CH(OH)—CH₂—CH₂—CH₂— | * | 2,6-Difluorophenyl | —CH₃ | 0 | Cl | 2.49 | |
| 467 | —CH₂—CH₂—C(CH₃)₂—CH₂—CH₂— | * | 2,6-Difluorophenyl | —CH₃ | 0 | Cl | 4.52 | |
| 468 | —CH₂—CH₂—CH(COCH₃)—CH₂—CH₂— | * | 2,6-Difluorophenyl | —CH₃ | 0 | Cl | 2.92 | |
| 469 | —CH₂—CH═C(C₂H₅)—CH₂—CH₂— | * | 2,6-Difluorophenyl | —CH₃ | 0 | Cl | 4.44 | |
| 470 | —CH₂—CH₂—CH═C(CH₃)—CH₂— | * | 2,6-Difluorophenyl | —CH₃ | 0 | Cl | 4.02 | |
| 471 | —CH(CH₂Cl)—CH₂—CH₂—CH₂— | * | 2,6-Difluorophenyl | —CH₃ | 0 | Cl | 3.23 | |
| | CH(CHCl)— | | | | | | | |
| 472 | —CH₂—CH₂—CH(COOCH₃)—CH₂—CH₂— | * | 2,6-Difluorophenyl | —CH₃ | 0 | Cl | 3.25 | |
| 473 | —CH₂—CH₂—CHBr—CH₂—CH₂— | * | 2,6-Difluorophenyl | —CH₃ | 0 | Cl | 3.91 | |
| 474 | —CH(COOCH₃)—CH₂—CH₂—CH₂—CH₂— | * | 2,6-Difluorophenyl | —CH₃ | 0 | Cl | 3.56 | |
| 475 | AB12 | * | 2,6-Difluorophenyl | —CH₃ | 0 | Cl | 3.71 | |
| 476 | —CH₂—CH₂—CH(NH—COCH₃)—CH₂— | * | 2,6-Difluorophenyl | —CH₃ | 0 | Cl | 2.18 | |
| | CH₂— | | | | | | | |
| 477 | —CH₂—CH₂—N(CH₃)—CH₂—CH₂— | * | 2,6-Difluorophenyl | —CH₃ | 0 | Cl | 1.4 | |
| 478 | AB14 | * | 2,6-Difluorophenyl | —CH₃ | 0 | Cl | 4.13 | |
| 479 | —CH₂—CH₂—CH₂—CH(CH₃)— | * | 2,4-Difluorophenyl | —CH₃ | 0 | Cl | 3.75 | |
| 480 | AB9 | * | 2,4-Difluorophenyl | —CH₃ | 0 | Cl | 1.47 | |
| 481 | —CH₂—CH₂—CH(OH)—CH₂—CH₂— | * | 2,4-Difluorophenyl | —CH₃ | 0 | Cl | 2.32 | |
| 482 | —CH₂—CH₂—CH₂—CH₂—CH(CH₃)— | * | 2,4-Difluorophenyl | —CH₃ | 0 | Cl | 4.26 | |
| 483 | —CH₂—CH₂—CH₂—CH(CH₃)—CH₂— | * | 2,4-Difluorophenyl | —CH₃ | 0 | Cl | 4.38 | |
| 484 | —CH₂—CH₂—C(CH₃)₂—CH₂—CH₂— | * | 2,4-Difluorophenyl | —CH₃ | 0 | Cl | 4.6 | |
| 485 | —CH₂—CH₂—CH(COCH₃)—CH₂—CH₂— | * | 2,4-Difluorophenyl | —CH₃ | 0 | Cl | 3.02 | |
| 486 | —CH₂—CH═C(C₂H₅)—CH₂—CH₂— | * | 2,4-Difluorophenyl | —CH₃ | 0 | Cl | 4.45 | |
| 487 | —CH₂—CH₂—CH═C(CH₃)—CH₂— | * | 2,4-Difluorophenyl | —CH₃ | 0 | Cl | 4.09 | |
| 488 | —CH₂—CH₂—CH(COOCH₃)—CH₂—CH₂— | * | 2,4-Difluorophenyl | —CH₃ | 0 | Cl | 3.32 | |
| 489 | —CH₂—CH₂—CH(CF₃)—CH₂—CH₂— | * | 2,4-Difluorophenyl | —CH₃ | 0 | Cl | 3.99 | |
| 490 | —CH₂—CH₂—N(CH₃)—CH₂—CH₂— | * | 2,4-Difluorophenyl | —CH₃ | 0 | Cl | 1.42 | |
| 491 | —CH₂—CH₂—CH₂—CH₂—CH₂— | * | 2,4-Difluorophenyl | —CH₃ | 0 | Cl | 3.97 | |
| 492 | —CH₂—CH₂—S—CH₂—CH₂— | * | 2,4-Difluorophenyl | —CH₃ | 0 | Cl | 3.53 | |
| 493 | —CH(CH₃)—CH₂—CH(CH₃)₂ | —H | 2,4-Difluorophenyl | —CH₃ | 0 | Cl | 4.19 | |
| 494 | —CH(CF₃)—CH₂—CH₂—CH₂— | * | 2,4-Difluorophenyl | —CH₃ | 0 | Cl | 4.12 | |
| 495 | —CH₂—CH₂—CH₂—CH₂— | * | 2,4-Difluorophenyl | —CH₃ | 0 | Cl | 3.45 | |
| 496 | —CH₂—CH₂—CH═CH—CH₂— | * | 2,4-Difluorophenyl | —CH₃ | 0 | Cl | 3.77 | |
| 497 | AB10 | * | 2,4-Difluorophenyl | —CH₃ | 0 | Cl | 3.33 | |
| 498 | —CH₂—CH(CH₃)—CH₂—CH(CH₃)—CH₂— | * | 2,4-Difluorophenyl | —CH₃ | 0 | Cl | 4.88 | |
| 499 | —CH₂—CH₂—CH(CH₃)—CH₂—CH₂— | * | 2,4-Difluorophenyl | —CH₃ | 0 | Cl | 4.46 | |
| 500 | —CH(CH₃)—CH₂—CH₂—CH₂—CH(CH₃)— | * | 2,4-Difluorophenyl | —CH₃ | 0 | Cl | 3.37 | |
| 501 | —CH₂—CH(OH)—CH₂—CH₂—CH₂— | * | 2,4-Difluorophenyl | —CH₃ | 0 | Cl | 2.65 | |
| 502 | —CH₂—CH₂—CHF—CH₂—CH₂— | * | 2,4-Difluorophenyl | —CH₃ | 0 | Cl | 3.51 | |
| 503 | —CH₂—CH(CH₃)—O—CH(CH₃)—CH₂— | * | 2,4-Difluorophenyl | —CH₃ | 0 | Cl | 3.63 | |
| 504 | —CH₂—CH₂—O—CH₂—CH₂— | * | 2,4-Difluorophenyl | —CH₃ | 0 | Cl | 2.93 | |
| 505 | i-Butyl | —H | 2,4-Difluorophenyl | —CH₃ | 0 | Cl | 3.67 | |
| 506 | —CH₂—C(CH₃)₃ | —H | 2,4-Difluorophenyl | —CH₃ | 0 | Cl | 4.07 | |
| 507 | 2-Butyl | —H | 2,4-Difluorophenyl | —CH₃ | 0 | Cl | 3.69 | |
| 508 | —CH₂—CH₂—CF₃ | —H | 2,4-Difluorophenyl | —CH₃ | 0 | Cl | 3.33 | |
| 509 | Cyclopentyl | —H | 2,4-Difluorophenyl | —CH₃ | 0 | Cl | 3.88 | |
| 510 | -i-Propyl | —H | 2,4-Difluorophenyl | —CH₃ | 0 | Cl | 3.36 | |
| 511 | Cyclohexyl | —H | 2,4-Difluorophenyl | —CH₃ | 0 | Cl | 4.16 | |
| 512 | 1-Cyclohexylethyl | —H | 2,4-Difluorophenyl | —CH₃ | 0 | Cl | 4.9 | |
| 513 | Cyclopropyl | —H | 2,4-Difluorophenyl | —CH₃ | 0 | Cl | 3.1 | |
| 514 | —CH₂—CF₃ | —H | 2,4-Difluorophenyl | —CH₃ | 0 | Cl | 3.19 | |
| 515 | Cyclopropylmethyl | —H | 2,4-Difluorophenyl | —CH₃ | 0 | Cl | 3.42 | |
| 516 | —CH₂—C(CH₃)═CH₂ | —H | 2,4-Difluorophenyl | —CH₃ | 0 | Cl | 3.34 | |
| 517 | 3-Trifluoromethylcyclohexyl | —H | 2,4-Difluorophenyl | —CH₃ | 0 | Cl | 4.14 | |
| 518 | 2-Trifluoromethylcyclohexyl | —H | 2,4-Difluorophenyl | —CH₃ | 0 | Cl | 4.18 | |
| 519 | 4-Trifluoromethylcyclohexyl | —H | 2,4-Difluorophenyl | —CH₃ | 0 | Cl | 4.11 | |
| 520 | —CH₂—CH₂—N(CH₃)₂ | —CH₃ | 2,4-Difluorophenyl | —CH₃ | 0 | Cl | 1.58 | |
| 521 | Propargyl | —CH₃ | 2,4-Difluorophenyl | —CH₃ | 0 | Cl | 3.29 | |
| 522 | —CH₂—COOC₂H₅ | —CH₃ | 2,4-Difluorophenyl | —CH₃ | 0 | Cl | 3.32 | |
| 523 | 1,3-Dioxolan-2-ylmethyl | —CH₃ | 2,4-Difluorophenyl | —CH₃ | 0 | Cl | 3.23 | |
| 524 | Allyl | —CH₃ | 2,4-Difluorophenyl | —CH₃ | 0 | Cl | 3.75 | |
| 525 | —CH₂—CH₂—CN | —CH₃ | 2,4-Difluorophenyl | —CH₃ | 0 | Cl | 2.84 | |
| 526 | —CH₂—CN | —CH₃ | 2,4-Difluorophenyl | —CH₃ | 0 | Cl | 2.84 | |
| 527 | (2-Furyl)methyl | —CH₃ | 2,4-Difluorophenyl | —CH₃ | 0 | Cl | 3.78 | |
| 528 | i-Butyl | —CH₃ | 2,4-Difluorophenyl | —CH₃ | 0 | Cl | 4.27 | |
| 529 | —CH₂—CH₂—Cl | —CH₃ | 2,4-Difluorophenyl | —CH₃ | 0 | Cl | 3.56 | |
| 530 | 2-Methoxyethyl | —CH₃ | 2,4-Difluorophenyl | —CH₃ | 0 | Cl | 3.27 | |
| 531 | —CH₂—C(CH₃)═CH₂ | —CH₃ | 2,4-Difluorophenyl | —CH₃ | 0 | Cl | 4.12 | |
| 532 | —CH₂—CH₂—N(CH₃)₂ | —C₂H₅ | 2,4-Difluorophenyl | —CH₃ | 0 | Cl | 1.71 | |
| 533 | Allyl | —C₂H₅ | 2,4-Difluorophenyl | —CH₃ | 0 | Cl | 4.09 | |
| 534 | (2-Tetrahydrofuryl)methyl | —C₂H₅ | 2,4-Difluorophenyl | —CH₃ | 0 | Cl | 3.95 | |
| 535 | —CH₂—CH₂—CN | —C₂H₅ | 2,4-Difluorophenyl | —CH₃ | 0 | Cl | 3.12 | |
| 536 | 2-Methoxyethyl | —C₂H₅ | 2,4-Difluorophenyl | —CH₃ | 0 | Cl | 3.57 | |
| 537 | —CH₂—COOC₂H₅ | —C₂H₅ | 2,4-Difluorophenyl | —CH₃ | 0 | Cl | 3.62 | |
| 538 | Cyclopropylmethyl | n-Propyl | 2,4-Difluorophenyl | —CH₃ | 0 | Cl | 4.65 | |
| 539 | (2-Tetrahydrofuryl)methyl | n-Propyl | 2,4-Difluorophenyl | —CH₃ | 0 | Cl | 4.32 | |
| 540 | 2-Methoxyethyl | n-Propyl | 2,4-Difluorophenyl | —CH₃ | 0 | Cl | 3.99 | |
| 541 | —CH₂—COOC₂H₅ | Cyclo- | 2,4-Difluorophenyl | —CH₃ | 0 | Cl | 3.87 | |
| | | propyl | | | | | | |
| 542 | —CH₂—CH(OH)—CH₂—CH₂— | * | 2,4-Difluorophenyl | —CH₃ | 0 | Cl | 2.33 | |
| 543 | 1,2-Dimethylpropyl | —H | 2,6-Difluorophenyl | —CH₃ | 0 | Cl | 3.83 | |
| 544 | 1,2-Dimethylpropyl | —H | 2-fluorophenyl | —CH₃ | 0 | Cl | 3.83 | |
| 545 | 2-Thienylinethyl | n-Propyl | 2,6-Difluorophenyl | —CH₃ | 0 | Cl | 4.61 | |
| 546 | —CH₂—COOC₂H₅ | Cyclo- | 2,6-Difluorophenyl | —CH₃ | 0 | Cl | 3.58 | |
| | | propyl | | | | | | |
| 547 | —CH₂—CH(NH₂)—CH₂—CH₂— | * | 2,6-Difluorophenyl | —CH₃ | 0 | Cl | 1.39 | |
| 548 | —CH₂—CH₂—CHF—CH₂—CH₂— | * | 2,6-Difluorophenyl | —CH₃ | 0 | Cl | 3.45 | |
| 549 | —CH₂—CH₂—CO—CH₂—CH₂— | * | 2,6-Difluorophenyl | —CH₃ | 0 | Cl | 2.68 | |
| 550 | —CH₂—CH₂—CH═CH—CH₂— | * | Pentafluorophenyl | —CH₃ | 0 | Cl | 4.2 | |
| 551 | AB10 | * | Pentafluorophenyl | —CH₃ | 0 | Cl | 3.86 | |
| 552 | —CH₂—CH(CH₃)—CH₂—CH(CH₃)—CH₂— | * | Pentafluorophenyl | —CH₃ | 0 | Cl | 5.31 | |
| 553 | —CH₂—CH₂—CH₂—CH₂—CH(CH₃)— | * | Pentafluorophenyl | —CH₃ | 0 | Cl | 4.83 | |
| 554 | —CH₂—CH₂—CH₂—CH(CH₃)—CH₂— | * | Pentafluorophenyl | —CH₃ | 0 | Cl | 4.89 | |
| 555 | —CH₂—CH₂—CH(CH₃)—CH₂—CH₂— | * | Pentafluorophenyl | —CH₃ | 0 | Cl | 4.88 | |
| 556 | —CH₂—CH(OH)—CH₂—CH₂—CH₂— | * | Pentafluorophenyl | —CH₃ | 0 | Cl | 3.14 | |
| 557 | —CH₂—CH₂—C(CH₃)₂—CH₂—CH₂— | * | Pentafluorophenyl | —CH₃ | 0 | Cl | 5.13 | |
| 558 | —CH₂—CH₂—CH(COCH₃)—CH₂—CH₂— | * | Pentafluorophenyl | —CH₃ | 0 | Cl | 3.6 | |
| 559 | —CH₂—CH═C(C₂H₅)—CH₂—CH₂— | * | Pentafluorophenyl | —CH₃ | 0 | Cl | 5 | |
| 560 | —CH₂—CH₂—CH═C(CH₃)—CH₂— | * | Pentafluorophenyl | —CH₃ | 0 | Cl | 4.6 | |
| 561 | —CH₂—CH₂—CH(COOCH₃)—CH₂—CH₂— | * | Pentafluorophenyl | —CH₃ | 0 | Cl | 3.89 | |
| 562 | —CH₂—CH₂—CHBr—CH₂—CH₂— | * | Pentafluorophenyl | —CH₃ | 0 | Cl | 4.5 | |
| 563 | —CH₂—CH₂—CF₂—CH₂—CH₂— | * | Pentafluorophenyl | —CH₃ | 0 | Cl | 4.12 | |
| 564 | AB12 | * | Pentafluorophenyl | —CH₃ | 0 | Cl | 4.36 | |
| 565 | AB11 | * | Pentafluorophenyl | —CH₃ | 0 | Cl | 1.91 | |
| 566 | —CH₂—CH₂—CH(NH—COCH₃)—CH₂— | * | Pentafluorophenyl | CH₃ | 0 | Cl | 2.79 | |
| | CH₂— | | | | | | | |
| 567 | AB14 | * | Pentafluorophenyl | —CH₃ | 0 | Cl | 4.67 | |

** The logP values were determined in accordance with EEC Directive 79/831 annex V. A8 by HPLC (gradient method, acetonitrile/0.1% aqueous phosphoric acid)
* R¹ and R² form a heterocyclic ring together with the nitrogen atom to which they are attached.
*** The abbreviations AB2, AB8, AB9, AB10, AB11, AB12, AB14 and AB29 have the following meanings:
AB2

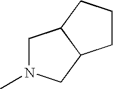

AB8

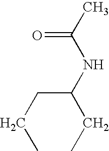

AB9

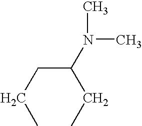

AB10

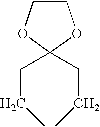

AB11

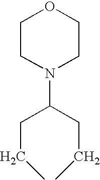

AB12

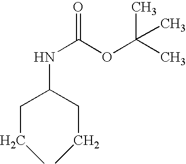

AB14

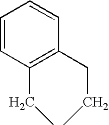

AB29

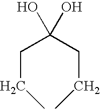

Preparation of Starting Materials

### Example 568

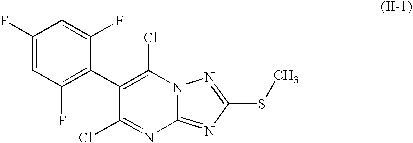

At room temperature, 31.3 g (95.35 mmol) of 2-methylsulphanyl-6-(2,4,6-trifluorophenyl)-[1,2,4]triazolo[1,5-a]pyrimidine-5,7-diol are dissolved in 140 ml of phosphorus oxychloride, 14.1 g of phosphorus pentachloride are added a little at a time and the mixture is then heated under reflux for 16 hours. Volatile components of the reaction mixture are distilled off under reduced pressure. 200 ml of water are added to the residue and the mixture is extracted with 100 ml of dichloromethane. The organic phase is dried over sodium sulphate and chromatographed on silica gel using initially ethyl acetate/n-hexane (1:3) and finally pure ethyl acetate. This gives 18 g (50% of theory) of 5,7-dichloro-2-(methylsulphanyl)-6-(2,4,6-trifluorophenyl)-[1,2,4]triazolo[1,5-a]pyrimidine.

HPLC: logP=3.43

The dihalogeno-triazolopyrimidines of the formula (II) listed in Table 2 below are also obtained by the method given above.

TABLE 2
(II)

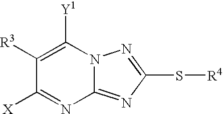

| Ex. No. | Comp. | R³ | R⁴ | X | Y¹ | logP | m.p. (° C.) |
|---|---|---|---|---|---|---|---|
| 569 | II-2 | 2-chlorophenyl | —CH₃ | —Cl | —Cl | | 159-164 |
| 570 | II-3 | 2-chlorophenyl | -i-propyl | —Cl | —Cl | | |
| 571 | II-4 | 2-chlorophenyl | 4-chlorobenzyl | —Cl | —Cl | | |
| 572 | II-5 | 2,6-dichlorophenyl | —CH₃ | —Cl | —Cl | 3.86 | |

### Example 573

Process (f), First Stage

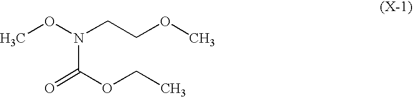

1 000 mg of ethyl N-methoxy-carbamate are introduced into 10.0 ml of dimethylformamide, and 403 mg of sodium hydride are added in portions, the temperature being adjusted to 30° C. by cooling. The reaction mixture is stirred at 30° C. for 2 hours and then 3 500 mg of 2-bromoethyl methyl ether are added. The reaction mixture is stirred at 20 to 25° C. for 18 hours and then stirred into 20 ml of water. The reaction mixture obtained is concentrated to dryness under reduced pressure and extracted with four times 30 ml of dichloromethane. The organic extracts are dried over sodium sulphate, filtered and concentrated to dryness under reduced pressure.

This gives 1 200 mg of ethyl (N-methoxy-N-methoxyethyl)carbamate (purity 77.6%, yield 62.6%).

Process (f), Second Stage:

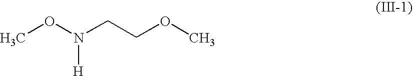

200 mg of ethyl (N-methoxy-N-methoxyethyl)-carbamate are introduced into 4.0 ml of aqueous ethanol (59%), 240.6 mg of potassium hydroxide are added and the reaction mixture is stirred at 40° C. for 18 hours. It is then stirred into 50 ml of water and extracted with three times 20 ml of diethyl ether and with three times 20 ml of dichloromethane. The combined organic phases are washed with twice 20 ml of water, dried and concentrated to a volume of 20 ml at 20° C. under reduced pressure. 2 ml of hydrochloric acid are added to the solution obtained, with ice cooling, and the solution is stirred at room temperature for one hour and concentrated to dryness at 20° C. under reduced pressure.

This gives 140 mg of N-methoxy-N-methoxyethylamine hydrochloride (yield 87.6%).

### Example 574

Process (g), First Stage:

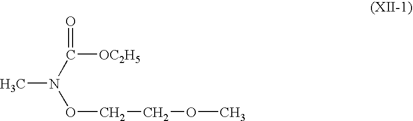

A mixture of 1 000 mg of ethyl N-hydroxy-N-methyl-carbamate, 1 166 mg of 2-bromoethyl methyl ether and 10 ml of ethanol is heated to reflux temperature with stirring and then a solution of 493 mg of potassium hydroxide in 5 ml of ethanol is added dropwise. The reaction mixture is boiled under reflux for 10 hours and then worked up by filtering the reaction mixture and concentrating the filtrate under reduced pressure. The residue is admixed with a mixture of water and ethyl acetate. The organic phase is separated off and washed with saturated aqueous ammonium chloride solution and then with water. The organic phase is subsequently dried over sodium sulphate and concentrated under reduced pressure. This gives 0.7 g of a product which according to the gas chromatogram consists of 83% of ethyl (N-methyl-N-methoxyethoxy)-carbamate. The yield is calculated accordingly to be 39% of theory.

Process (g), Second Stage:

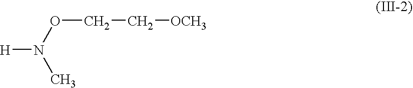

240.6 mg of powdered potassium hydroxide are added to a mixture of 200 mg of ethyl (N-methyl-N-methoxyethoxy)-carbamate, 4 ml of ethanol and 4 ml of water and the reaction mixture is stirred at 40° C. for 42 hours. It is subsequently stirred into 50 ml of water and then extracted with three times 20 ml of diethyl ether and then with three times 20 ml of methylene chloride. The combined organic phases are washed with twice 20 ml of water, dried over sodium sulphate and concentrated to a volume of 20 ml at room temperature under reduced pressure. The solution obtained is admixed with 1 ml of ethereal hydrochloric acid, with ice cooling. The crystals which deposit are filtered off and dried. This gives 190 mg of N-methyl-N-methoxyethoxyamine hydrochloride.

### Example 575

Process (h), First Stage:

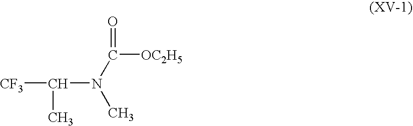

475 mg of sodium hydroxide are added at room temperature with stirring to a mixture of 2 000 mg of ethyl N-(2,2,2-trifluoro-1-methylethyl)-carbamate and 20 ml of tetrahydrofuran. Thereafter a solution of 4 600 mg of iodomethane in 10 ml of tetrahydrofuran is added dropwise at room temperature with stirring. The reaction mixture is stirred at 50° C. for 16 hours and then water is added. The mixture is extracted with three times 20 ml of methylene chloride and the combined organic phases are dried over sodium sulphate and concentrated under reduced pressure. This gives 1 000 mg of a product which according to the gas chromatogram consists of 75% of ethyl N-(2,2,2-trifluoro-1-methyl-ethyl)-N-methyl-carbamate. The yield is calculated accordingly to be 34.86%.

Process (h), Second Stage:

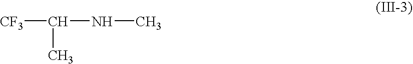

1 070 mg of powdered potassium hydroxide are added to a mixture of 1 000 mg of ethyl N-(2,2,2-trifluoro-1-methylethyl)-N-methylcarbamate, 20 ml of ethanol and 20 ml of water and the mixture is stirred at 40° C. for 66 hours. The reaction mixture is subsequently diluted with water and extracted with three times 20 ml of a mixture consisting of equal parts of methylene chloride and diethyl ether. The combined organic phases are dried over sodium sulphate and then concentrated at room temperature under slightly reduced pressure. Ethereal hydrochloric acid is added to the resulting solution, with ice cooling, and the solution is stirred at room temperature for 60 hours. Concentration under reduced pressure gives 280 mg of N-(2,2,2-trifluoro-1-methyl-ethyl)-N-methylamine hydrochloride. The yield is calculated accordingly to be 34% of theory.

### Example 576

Process (i):

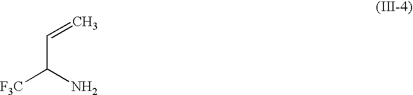

600 mg of benzyl N-(1-trifluoromethyl-2-propene)-carbamate are heated under reflux in 8.0 ml of 16% strength hydrochloric acid for 1.5 hours. After the reaction mixture has cooled to 20° C. it is extracted with twice 20 ml of diethyl ether.

The aqueous phase remaining is concentrated to dryness under reduced pressure and three times 10 ml of methanol are added. After removal of the methanol under reduced pressure, 310 mg of (1-trifluoromethyl-prop-2-ene)-amine hydrochloride are isolated. The yield is calculated accordingly to be 82.9% of theory.

In accordance with the methods indicated above it is also also possible to prepare the carbamates indicated in the following tables.

TABLE 3
(X)

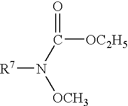

| Example No. | Comp. No. | R⁷ | logP |
|---|---|---|---|
| 577 | X-2 | 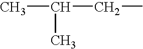 | 2.38 |
| 578 | X-3 | 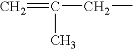 | 2.06 |

TABLE 4

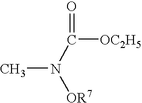

| Example No. | Comp. No. | R⁷ | Physical constants |
|---|---|---|---|
| 579 | XII-22 | 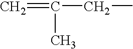 | |

TABLE 5
(XV)

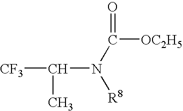

| Example No. | Comp. No. | R⁸ | Physical constants |
|---|---|---|---|
| 580 | XV-2 | —C₂H₅ | ¹H-NMR (400MHz, CD₃CN): |
| | | | δ(ppm) = 1.13(t, CH₃CH₂N), |
| | | | 1.21(t, CH₃CHCF₃), 1.23(t, |
| | | | CH₃CH₂O), 3.20(m, CH₂N, |
| | | | CHCF₃), 4.1(q, CH₃CH₂O). |

In accordance with the methods indicated above it is also possible to prepare the amines listed in the following table.

TABLE 6
(III)

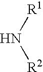

| Ex. | Comp. | | | |
|---|---|---|---|---|
| No. | No. | R¹ | R² | Physical constants |
| 581 | III-5 | 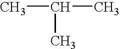 | —OCH₃ | ¹H-NMR(400 MHz, CD₃CN): δ(ppm) = 1.03(d, (CH₃)₂CH), 3.06(d, CH₂), 3.28(b, (CH₃)₂CH), 4.01(s, OCH₃) |
| 582 | III-6 | 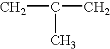 | —OCH₃ | ¹H-NMR(400 MHz, DMSO): δ(ppm) = 1.76(s, CH₃—(CCH₂)CH₂), 3.29(b, NH, CH₃(CCH₂)CH₂, OCH₃), 7.89, 5.02(2s, CH₃(CCH₂)CH₂). |
| 583 | III-7 | 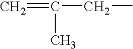 | —CH₃ | |
| 584 | III-8 | 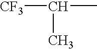 | —C₂H₅ | ¹H-NMR(400 MHz, DMSO): δ(ppm) = 1.06(m, CH₃CH₂N, CH₃CHCF₃), 3.20(m, CH₂N), 4.1(m, CHCF₃) |

The amines listed in Examples 581-584 were each isolated and characterized in the form of their hydrochlorides.

### Example 585

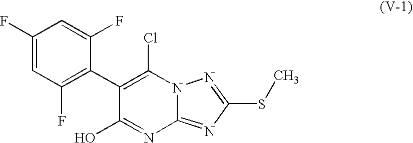

5 g (19.1 mmol) of dimethyl 2-(2,4,6-trifluoro-phenyl)malonate and 2.5 g (19.1.mmol) of 5-methylsulphanyl-1H-[1,2,4]triazol-3-ylamine in 5 ml of tri-n-butylamine are heated at 180° C. for 6 hours, during which the methanol that is formed is distilled off. The tri-n-butylamine is distilled off under strongly reduced pressure. This gives 7.9 g (99.7% of theory) of crude 79% 2-methylsulphanyl-6-(2,4,6-trifluoro-phenyl)-[1,2,4]triazolo[1,5-a]pyrimidine-5,7-diol, which is reacted further without purification.

HPLC: logP=0.47

### USE EXAMPLES

### Example A

Podosphaera Test (Apple)/Protective

| | |
|---|---|
| Solvent: | 24.5 parts by weight of acetone |
| | 24.5 parts by weight of dimethylacetamide |
| Emulsifier: | 1.0 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of the apple mildew pathogen Podosphaera leucotricha. The plants are then placed in the greenhouse at about 23° C. and a relative atmospheric humidity of about 70%.

Evaluation is carried out 10 days after the inoculation. 0% means an efficacy which, corresponds to that of the control, whereas an efficacy of 100% means that no infection is observed.

Active compounds, application rates and results of the experiments are shown in the following table:

TABLE A
Podosphaera test (apple)/protective

| | Application rate of | |
|---|---|---|
| Active compound | active compound in g/ha | Efficacy in % |
| Inventive: | | |
| 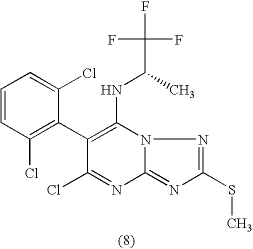 | 100 | 100 |
| 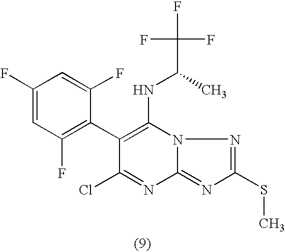 | 100 | 94 |
| 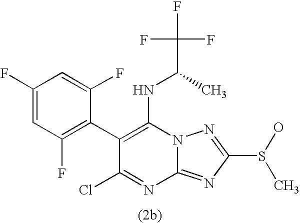 | 100 | 98 |

### Example B

Venturia Test (Apple)/Protective

| | |
|---|---|
| Solvent: | 24.5 parts by weight of acetone |
| | 24.5 parts by weight of dimethylacetamide |
| Emulsifier: | 1.0 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for one day.

The plants are then placed in a greenhouse at about 21° C. and a relative atmospheric humidity of about 90%.

Evaluation is carried out 12 days after the inoculation. 0% means an efficacy which corresponds to that of the control, whereas an efficacy of 100% means that no infection is observed.

Active compounds, application rates and results of the experiments are shown in the following table.

TABLE B
Venturia-Test (apple)/protective

| | Application rate of | |
|---|---|---|
| Active compound | active compound in g/ha | Efficacy in % |
| Inventive: | | |
| 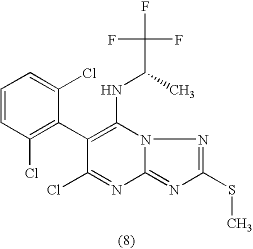 | 100 | 100 |
| 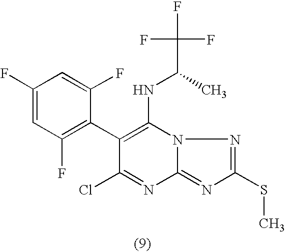 | 100 | 100 |
| 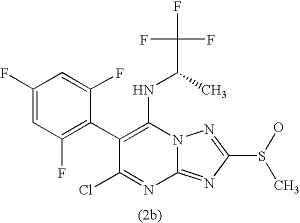 | 100 | 100 |

### Example C

In Vitro Test for Determining the ED₅₀ on Microorganisms

| | |
|---|---|
| Solvent: | 1 ml of methanol |
| Emulsifier: | 6 mg of ethoxylated tristyrylphenol |

To produce a suitable preparation of active compound, 20 μg of active compound are mixed with the amounts of solvent and emulsifier stated above, and the concentrate is diluted with the solvent/emulsifier mixture stated above to the concentration desired in each case.

In each case 10 μl of the preparation are pipetted into the wells of microtitre plates. After the solvent has evaporated, in each case 200 μl of a potato dextrose medium which had earlier been admixed with the respectively desired concentration of spores or mycelium of the microorganism to be tested are added to each well. The resulting concentrations of active compound in the wells are

| | |
|---|---|
| 0.1 | ppm |
| 1 | ppm |
| 10 | ppm |
| and 100 | ppm. |

The resulting concentrations of emulsifier are in each case 300 ppm.

For incubation, the microtitre plates are then agitated on a shaker at a temperature of 22° C. for 3 to 5 days until sufficient growth of the microorganism in question can be detected in the untreated control.

Evaluation is carried out photometrically at a wavelength of 620 nm. From the data measured for the various concentrations, the dose of active compound which results in 50% inhibition of fungal growth (ED₅₀), compared to the untreated control, is calculated.

Active compounds and results of the experiments are shown in the following table.

TABLE C
In vitro test for determining ED₅₀ in microorganisms

| Active compound | Microorganism | ED₅₀ |
|---|---|---|
| Inventive: | | |
| 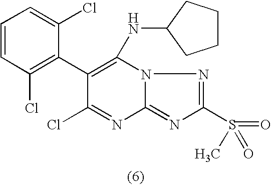 | Pyricularia oryzae | 0.24 |
| 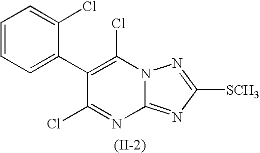 | Pyricularia oryzae | 0.33 |

1. Triazolopyrimidines of the formula 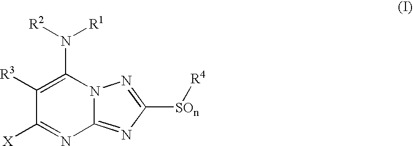
in which
R¹ represents amino, optionally substituted alkyl, optionally substituted alkenyl, optionally substituted alkinyl, optionally substituted cycloalkyl, optionally substituted alkoxy, optionally substituted alkenyloxy, optionally substituted alkinyloxy, optionally substituted cycloalkyloxy, optionally substituted alkylamino, optionally substituted dialkylamino, optionally substituted alkenylamino, optionally substituted alkinylamino, optionally substituted cycloalkylamino, optionally substituted N-cycloalkyl-N-alkylamino, optionally substituted alkylideneamino, optionally substituted heterocyclyl or represents a radical of the formula —S—R⁵, in which
R⁵ represents optionally substituted alkyl, optionally substituted alkenyl, optionally substituted alkinyl or optionally substituted cycloalkyl,
R² represents hydrogen, optionally substituted alkyl, optionally substituted alkenyl, optionally substituted alkinyl, or optionally substituted cycloalkyl,
or
R¹ and R² together with the nitrogen atom to which they are attached represent an optionally substituted heterocyclic ring,
R³ represents optionally substituted aryl,
R⁴ represents optionally substituted alkyl, optionally substituted alkenyl or optionally substituted alkinyl,
X represents halogen and
n represents 0, 1 or 2,
and acid addition salts of those compounds of the formula (I) in which
R¹ represents amino.

2. Process for preparing triazolopyrimidines of the formula (I) according to claim 1, characterized in that
a) dihalogeno-triazolopyrimidines of the formula 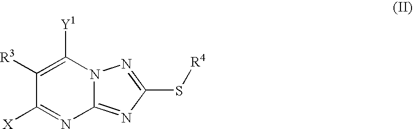
in which
R³, R⁴ and X have the meanings given above and
Y¹ represents halogen
are reacted with amines of the formula 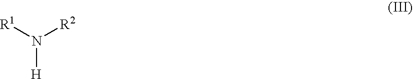
in which
R¹ and R² have the meanings given above,
if appropriate in the presence of a diluent and if appropriate in the presence of an acidic acceptor,
or
b) triazolopyrimidines of the formula 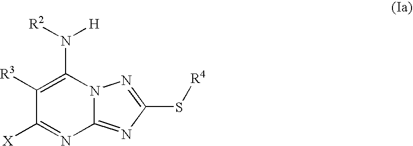
in which
R², R³, R⁴ and X have the meanings given above
are reacted with sulphenyl halides of the formula
Y²—S—R⁵ (IV)
in which
R⁵ has the meanings given above and
Y² represents halogen
if appropriate in the presence of a diluent and if appropriate in the presence of an acid acceptor,
or
c) triazolopyrimidines of the formula 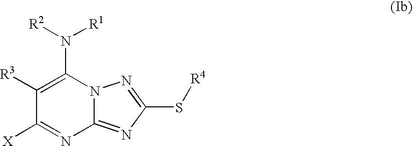
in which
R¹, R², R³, R⁴ and X have the meanings given above, are reacted with oxygen-releasing oxidizing agents, if appropriate in the presence of a diluent and if appropriate in the presence of a catalyst,
and if appropriate adding an acid to the resulting compounds of the formula (I) in which
R¹ represents amino.

3. Compositions for controlling unwanted microorganisms, characterized in that they comprise at least one triazolopyrimidine of the formula (I) according to claim 1 or an acid addition salt thereof, in addition to extenders and/or surfactants.

4. Use of triazolopyrimidines of the formula (I) according to claim 1, or their acid addition salts, for controlling unwanted microorganisms.

5. Method for controlling unwanted microorganisms, characterized in that triazolopyrimidines of the formula (I) according to claim 1 or their acid addition salts are applied to the unwanted microorganisms and/or their habitat.

6. Process for preparing compositions for controlling unwanted microorganisms, characterized in that triazolopyrimidines of the formula (I) according to claim 1 or their acid addition salts are mixed with extenders and/or surfactants.

7. Amines of the formula 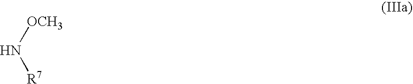
in which
R⁷ represents isobutyl, 2-methoxyethyl or represents

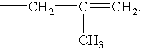

8. Process for preparing amines of the formula (IIIa) according to claim 7, characterized in that
f) in a first stage ethyl N-methoxy-carbamate of the formula 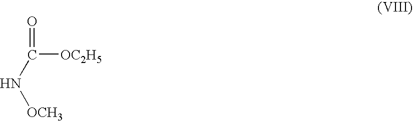
is reacted with halogen compounds of the formula
R⁷—X¹ (IX)
in which
R⁷ has the meanings given above and
X¹ represents bromine or iodine
in the presence of a base and in the presence of a diluent and in a second stage the resulting carbamates of the formula 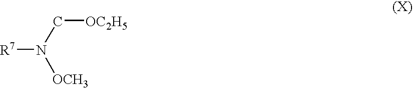
in which
R⁷ has the meanings given above
are reacted with potassium hydroxide in the presence of ethanol and water.

9. Amines of the formula 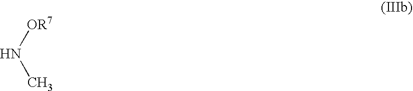
in which
R⁷ represents isobutyl, 2-methoxyethyl or represents

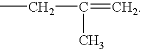

10. Process for preparing amines of the formula (IIIb) according to claim 9, characterized in that
g) in a first stage ethyl N-hydroxy-N-methyl-carbamate of the formula 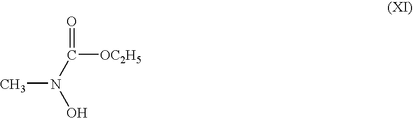
is reacted with halogen compounds of the formula
R⁷—X¹ (VIII)
in which
R⁷ and X¹ have the meanings given above
in the presence of a base and in the presence of a diluent and in a second stage the resulting carbamates of the formula 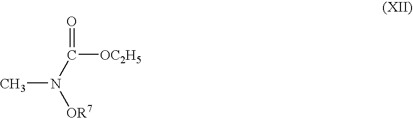
in which
R⁷ has the meanings given above
are reacted with potassium hydroxide in the presence of ethanol and water.

11. Trifluoroisopropylamines of the formula 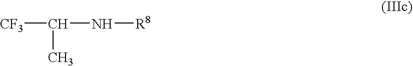
in which
R⁸ represents methyl, ethyl or propyl.

12. Process for preparing trifluoroisopropylamines of the formula (IIIc) according to claim 11, characterized in that
h) in a first stage ethyl N-trifluoro-isopropylcarbamate of the formula 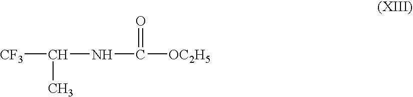
is reacted with halogen compounds of the formula
R⁸—X¹ (XIV)
in which
R⁸ and X¹ have the meanings given above
in the presence of a base and in the presence of a diluent and in a second stage the resulting carbamate of the formula 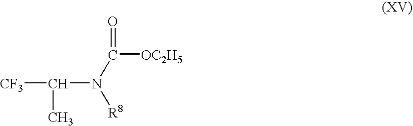
in which
R⁸ has the meanings given above
are reacted with potassium hydroxide in the presence of ethanol and water.

13. 3-Trifluoromethyl-3-amino-propene of the formula

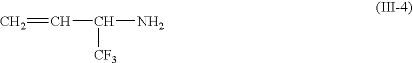

14. Process for preparing 3-trifluoromethyl-3-amino-propene of the formula (III-4) according to claim 13, characterized in that
i) the carbamate of the formula 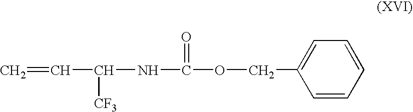
is reacted with aqueous hydrochloric acid.

15. Carbamates of the formula 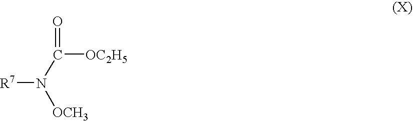
in which
R⁷ represents isobutyl, 2-methoxyethyl or represents

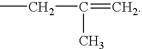

16. Carbamates of the formula 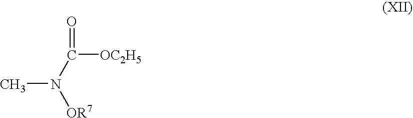
in which
R⁷ represents isobutyl, 2-methoxyethyl or represents

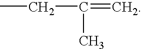

17. Carbamates of the formula 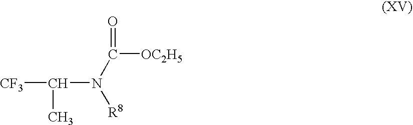
in which
R⁸ represents methyl, ethyl or propyl.
